(19) United States
(12) Patent Application Publication
(10) Pub. No.: US 2017/0319652 A1
(43) Pub. Date: Nov. 9, 2017

(54) PREVENTION AND TREATMENT OF INFECTIONS

(71) Applicants: ImmunoClin Limited, London (GB); Institut De Recherche Pour Le Developpment (IRD), Paris (FR)

(72) Inventors: Francisco Veas, Mauguio (FR); Dorothee Misse, Montpellier (FR); Mario Clerici, Milano (IT); Daria Trabatoni, Desio (IT)

(73) Assignee: ImmunoClin Limited, London (GB)

(21) Appl. No.: 15/257,795

(22) Filed: Sep. 6, 2016

Related U.S. Application Data
(63) Continuation-in-part of application No. 11/381,811, filed on May 5, 2006, now abandoned.
Application No. 11/381,811 is a continuation-in-part of application No. PCT/EP04/13393, filed on Nov. 5, 2004.
(60) Provisional application No. 60/517,104, filed on Nov. 5, 2003.
(60) Provisional application No. 60/580,720, filed on Jun. 21, 2004.

(51) Int. Cl. A61K 38/17; A61K 38/19; A61K 38/20; G01N 33/569

(52) U.S. Cl. CPC A61K 38/1709; G01N 33/56988; A61K 38/195; A61K 38/20; C12Q 2600/158

(57) ABSTRACT

The invention relates to the use of IL-22 in prevention and therapy of infections in particular related to viral infections in individuals who are unable to respond to viral exposure with enhanced IL-22 gene expression or protein production.

The present application is a continuation-in-part of U.S. patent application Ser. No. 11/381,811, filed May 5, 2006, which is a continuation-in-part of International Application No. PCT/EP04/013393, filed Nov. 5, 2004, which designated the U.S., the present application also claims benefit of U.S. Provisional Application Ser. Nos. 60/673,340; U.S. 60/517,104 and 60/5S0,720, filed Apr. 21, 2005, Nov. 5, 2003 and Jun. 21, 2004, respectively; the entire contents of each of which is hereby incorporated herein by reference.

The invention concerns biomarkers of resistance to infections in humans and biological applications thereof, particularly in diagnostics, prophylaxis and therapeutics.

It relates to biomarkers of resistance to infections due to pathogens in general, particularly infections due to viruses.

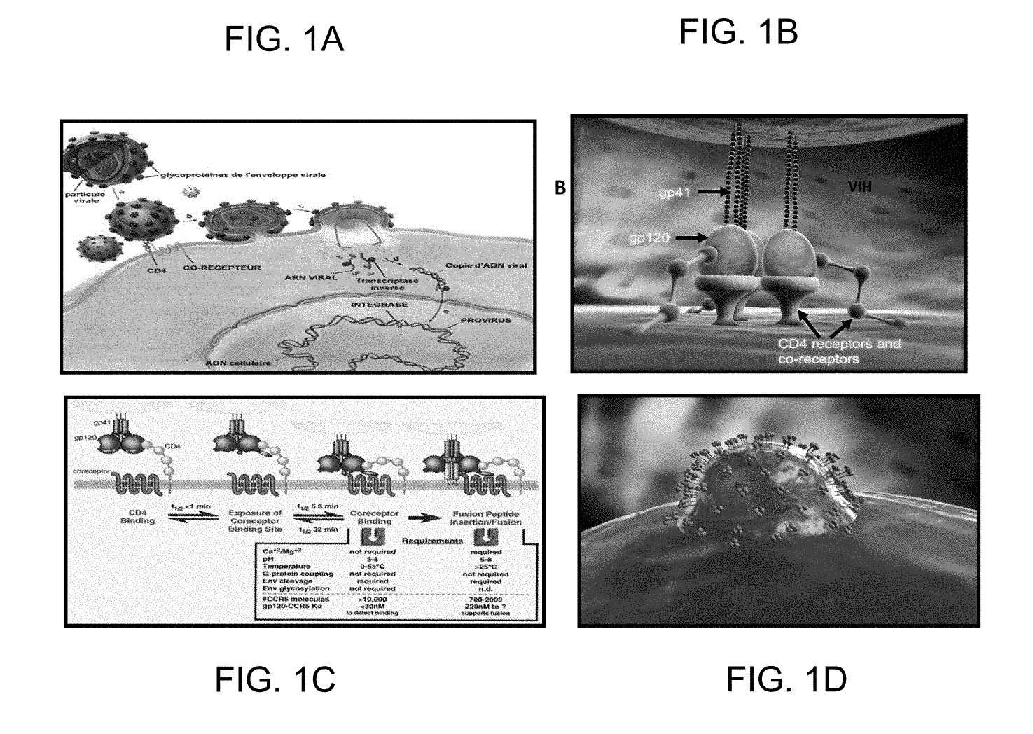

Several viral diseases emerged at the end of the twentieth century, particularly the Acquired Immunodeficiency Syndrome (AIDS) caused by the human immunodeficiency virus (HIV). More than two decades since its discovery, human immunodeficiency virus (HIV) epidemic is still a major burden for health, social and economic reasons on all over the world. To date, both host genetic repertoire, innate and acquired immune responses, viral mutation or attenuation have been invoked to explain the higher or lower individual susceptibility to the infection. A great deal of progress has been made in understanding the mechanism of human immunodeficiency virus entry into target cells. Landmark discoveries such as the identification of viral co-receptors and the structure of the viral envelope protein (Env) bound to its receptor provided important insight into how Env mediates fusion of the viral and cellular membranes as described in FIGS. 1A to 1D.

The existence of some people somewhat “immune” from infection, despite dealing with repeated HIV exposure, as well as the extremely slow disease progression in some HIV infected individuals, offers valuable clues to elucidate mechanisms underlying natural HIV resistance. Strikingly, both such cohorts, the so-called Exposed Seronegative, Exposed Uninfected (ESN, EU) and the Slow Progressors, Long Term Progressors (SP, LTNP) individuals have common immune responses, e.g. the generation of neutralizing antibodies directed against common targets, which can play a protective role in virus entry and/or spread.

In 1989, Ranki et al described a curious phenomenon: HIV-specific T-cell response to HIV, native gp 120 and recombinant envelope and core proteins could be detected in antibody- and antigen-negative sexual partners of known HIV-positive men [1]. Two other reports confirmed that initial observation, and the authors raised the possibility that exposure to HIV that did not result in seroconversion and infection would be associated with the exclusive priming of T helper lymphocytes [2,3]. Analyses performed in different cohorts of individuals at high risk of HIV infection, and including health care workers parenterally exposed to HIV and healthy newborns of HIV-infected mothers, revealed that HIV-specific T helper cells, but not antibodies, were present in all these subjects [4]. These observations led to the hypothesis that viral exposure resulting in the exclusive priming of HIV-specific T cells could be associated with protection against actual HIV infection. This hypothesis was greatly strengthened by three commercial sex workers in Narobi [5] (the Pumwaani cohort), clearly demonstrated that whereas the majority of women who started to prostitute themselves became HIV infected within a year, a sizable minority, subsequently estimated to be around 15% of the individuals tested, was clearly resistant to infection. 2) Sarah Rowland-Jones [6] showed the presence of HIV-specific CTL in healthy newborns of HIV infected mothers. The detection of HIV-specific, IFNα-secreting CD8 T lymphocytes in these newborns was a turning point in the realization that HIV exposure not associated with seroconversion is associated with an actual abortive infection and that live, replicating virus is indeed responsible for the stimulation of specific immunity. In fact, only actual infection with the virus would result in presentation of viral antigens in association with HLA class I molecules, and elicitation of a CD8-mediated immune response. (much later, the protective role of cell mediated immunity in this setting was further reinforced by the observation that late seroconversion occurring in Kenyan HIV-resistant sex workers who interrupt commercial sex work for a period of time is related to the waning of HIV-specific CD8+responses due to reduced antigenic exposure) [7]. 3) Experiments in which macaques exposed in vivo to subinfectious doses of SIV, and in whom SIV-specific T helper cells were detected, demonstrated protection against subsequent challenges with infectious doses of the same virus [8] (these results were not unequivocally confirmed by other investigators).

The field of investigation of immune correlates of protection against HIV infection was born. Subsequent, pivotal reports showed that in HIV-exposed but uninfected individuals: 1) a particular genetic background, epitomized by the 0.32 deletion in the CCR5 receptor [9], could be present [10-12]; 2) the production of soluble factors, including cell antiviral factors (CAF) [13,14], beta chemokines, and alpha defensins [15], is increased [16-IS]; 3) secretory HIV-specific IgA as well as T helper and CTL can be detected in cervico-vaginal fluids and ejaculates[19-22]; and 4) NK cell activity is particularly potent [23]. Thus, 15 years after the first description of the detection of HIV-specific T helper cells in seronegative individuals, possible resistance to HIV infection can be summarized as being correlated with the elicitation of systemic and mucosa! cell mediated immunity, and mucosally-confined IgA, possibly within favourable genetic and natural immunity settings.

Mechanisms suggested to be associated with resistance to HIV infections are summarized in Table 1 below.

TABLE I

| Acquired mechanisms | Genetic mechanisms | Innate Immunity |
|---|---|---|
| HIV-specific T helper cells | Deletion in the HIV-I | Elevated NK activity |
| HTV-specific CTL | Co-receptors | Elevated production of β |
| Mucosal HIV-specific IgA | Particular HLA alleles | chemokines |
| Anti CD4 antibodies | | Elevated production of CAF |
| Anti CCR5 antibodies | | Elevated concentration of α |
| | | defensins |

The comprehension of mechanisms of natural resistance to HIV infection may have implications for the identification of anti-viral novel strategies and in particular for the development of innovative diagnostics, therapeutics and vaccine design.

The inventors have compared studies on protein profiles (proteom) and genome expression (transcriptome) from HIV exposed uninfected individuals (EU), HIV exposed and infected individuals (HIV+) and healthy donors (HC) to identify biomarkers from EU that could explain resistance mechanisms to the HIV infection.

They have identified a key pro-inflammatory cytokine IL-22 which appears to be responsible for the induction of proteins involved in a more global innate immune response that contributes to the viral resistance, including proteins that are produced from genes, and more particularly for the induction of an innate immunity pathway that can be stimulated by any pathogenic antigens, particularly any virus, to achieve protective immunity against the antigens, notably viruses.

They have also found that IL-22 induced acute phase proteins such as A-SAA (1 or 2) shows a polymorphism among the studied cohorts exhibiting a particular pattern in EU. Some of these isoforms also appear to be involved in HIV resistance processes by their effect on FPR or FPRL1 receptors and the subsequent phosphorylation of CCR5 or CXCR4 HIV-co-receptors.

They have also shown in vitro in ectocervical epithelia cells that IL-22 induced beta-defensins 2 and 3, but not beta-defensin 1. Then the combination of these proteins was considered as element participating indirectly to the viral infection blockade. Individually and in combination, they also appear to participate in the HIV resistance mechanisms.

It is then the objective of the invention to provide biomarkers of resistance to HIV-infections.

According to another objective, the invention aims to provide new tools useful in diagnostics, prophylaxis and therapeutics comprising the use individually or in combination, of said proteins and the proteins inducing said cascade.

It also relates to the use of viral antigens in a form of attenuated virus particles or an antigenic part thereof as protective and therapeutic products.

Said virus antigens can be used in combination with cytokines.

Said virus comprises for example hepatitis viruses, respiratory viruses and HIV viruses.

The invention more specifically relates to the use of IL-22 as biomarkers of the resistance to viral infections, particularly HIV infection, when measured as gene expression or as level of cytokine.

As illustrated in the examples, the inventors have identified that IL-22 triggers a biochemical cascade of events and provided evidence that IL-22 has a pivotal role in the innate resistance to viral infections and more specifically HIV-1 infection and influenza infection. Results shown in the examples indicate that IL-22 not only induces acute phase proteins such as A-SAA and β-defensins, but also initiates the A-SAA-mediated production of IL-16, resulting in phosphorylation and down-regulation of CCR5 and, finally, in a decreased in vitro susceptibility of target cells to infection with primary isolates of HIV. These mechanisms are likely to be important in the development of novel therapeutic and vaccination approaches for HIV infection. The connection of the multiple elements of the cascade to pathology in viral diseases including HIV have been shown by others but the fact IL-22 is the initiating trigger of such cascade is a surprising discovery.

Advantageously, the biomarkers comprise IL-22 and one or several of the proteins selected in the group comprising SOCS1, and/or STAT3, a soluble protein of about 8.6 kDa as identified in plasmas by SELDI-TOF.

Proteins from the Jack/STAT and/or SOCS axis are phosphorylated According to an embodiment of the invention, the biomarkers of chemokines comprise, in addition to IL-22 or IL-22 and one or several of said proteins, proteins selected in the group comprising GRO-α, MIP-3β, SDF1-β, and/or the gamma chemokine lymphotactin and/or isoforms thereof.

Said proteins are of great value in biological applications in view of their properties as biomarkers of resistance to viral infections, particularly HIV infections. They are of great interest in diagnostics, therapeutics and prophylaxis.

The invention thus also relates to their use as diagnostic tools comprising using said proteins.

The invention also relates to pharmaceutical compositions for preventing or treating any infection due to pathogens, particularly viral or retro-viral infections, more particularly HIV and influenza infections.

Such compositions comprise an effective amount of IL-22, optionally in combination with at least one of the above proteins defined as biomarkers and are useful as drugs. The invention also relates to pharmaceutical composition comprising an effective amount of IL-22, optionally in combination with at least one of the above proteins defined as biomarkers in association with a pharmaceutically acceptable carrier. Such pharmaceutical compositions comprise an effective amount of IL-22 in a form of cytokine or encoding DNA in association with a pharmaceutically inert vehicle.

The pharmaceutical compositions of the invention are advantageously prepared for administration by the oral, or mucosa! route, or for injection.

For oral administration, they are presented in the form of tablets, pills, capsules, drops, patch or spray.

For administration by injection, the pharmaceutical compositions are under the form of solution for injection by the intravenous, subcutaneous or intramuscular route produced from sterile or sterilisable solution, or suspension or emulsion.

For administration by mucosa! route, the pharmaceutical compositions are under the form of gels.

The administration doses will easily be adjusted by the one skilled in the art depending on the patient's condition.

In still another aspect, the invention relates to a multifactorial innate immunity assessment method, comprising the use of IL-22 in research diagnostic products.

According to still another aspect, the invention relates to a method for favouring the innate host resistance to viral infections, comprising using IL-22 as starter cytokine helping the innate immune response to infections, wherein IL-22 is used as a prophylactic agent in triggering the immune response to viral infections, particularly HIV infections.

According to still a further aspect, the invention relates to a method for favouring the innate host resistance to viral infections, comprising using IL-22 as starter cytokine helping the innate immune response to infections, wherein IL-22 is used as a therapeutic agent in triggering the immune response to viral infections, particularly HIV infections particularly HIV infections.

Optionally, IL-22 is used with at least one of the proteins above defined as biomarkers.

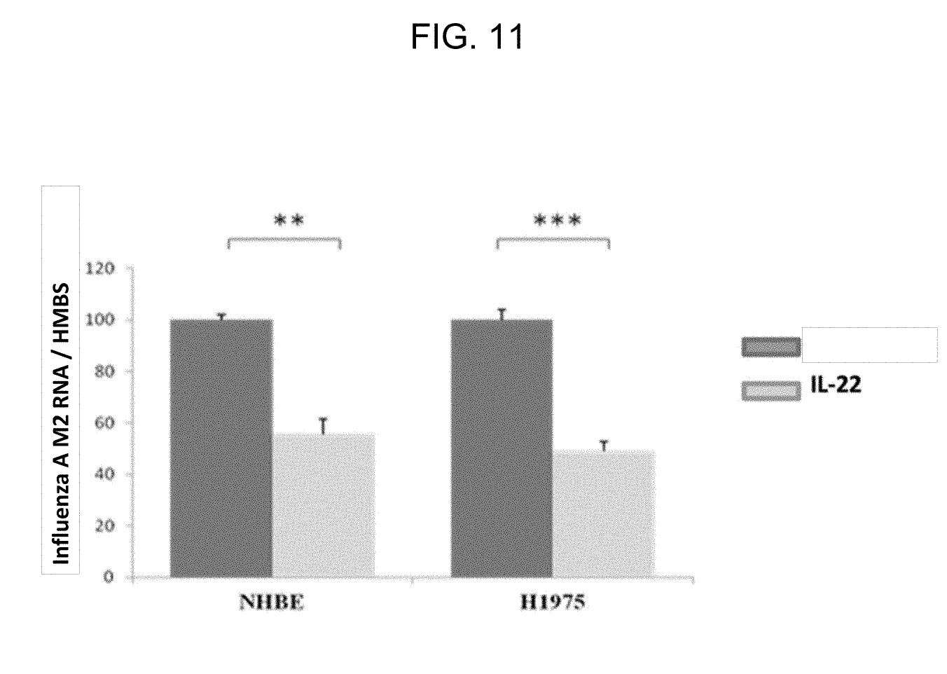

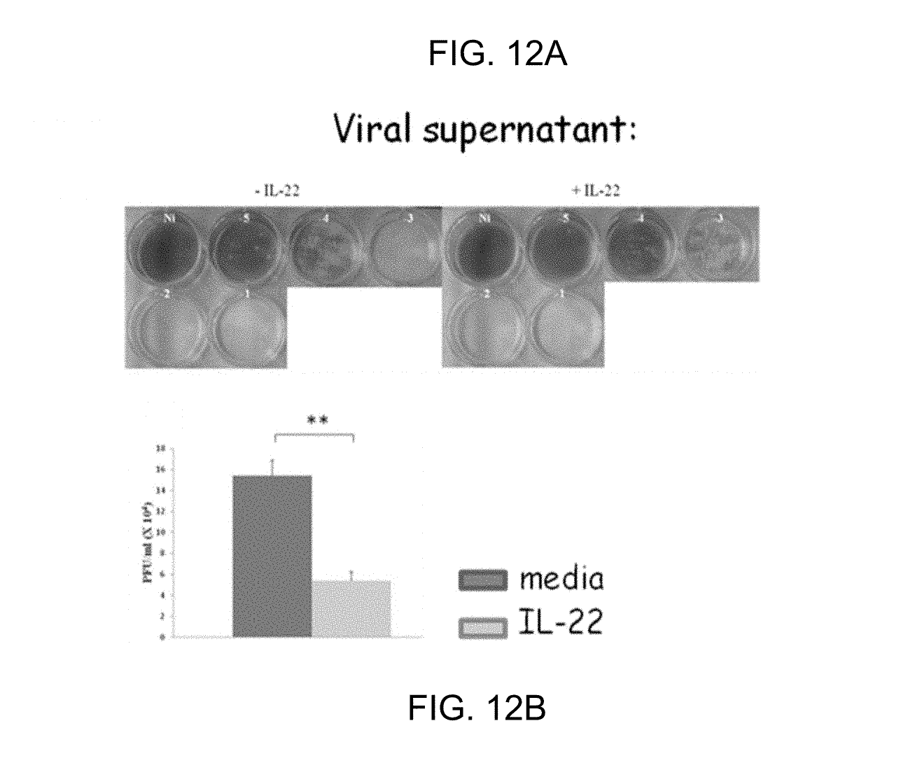

The invention also relates to the use of IL-22 as treatment of influenza infection. The inventors have evaluated the role of IL-22 in multiple models of H1 N1 viral infection. In vitro data obtained using lung cells lines (A549, H1975) and primary cell cultures NHBE (Lonza) have shown that IL-22 administration reduces viral infection (H1 N1 (WSN) and H3N2 (A/Pan/2007/99). To study the effect of IL-22 on viral entry, quantifications of viral RNA have been done 5 h after infection of NHBE primary cell cultures and H1975 cell line with H1 N1 (WSN) (FIG. 11). Effect of IL-22 has been evaluated 48 hr after infection by quantification of PFU in corresponding supernatants (FIGS. 12A and 12B). The data show that IL-22 inhibits influenza viral entry and influenza viral replication.

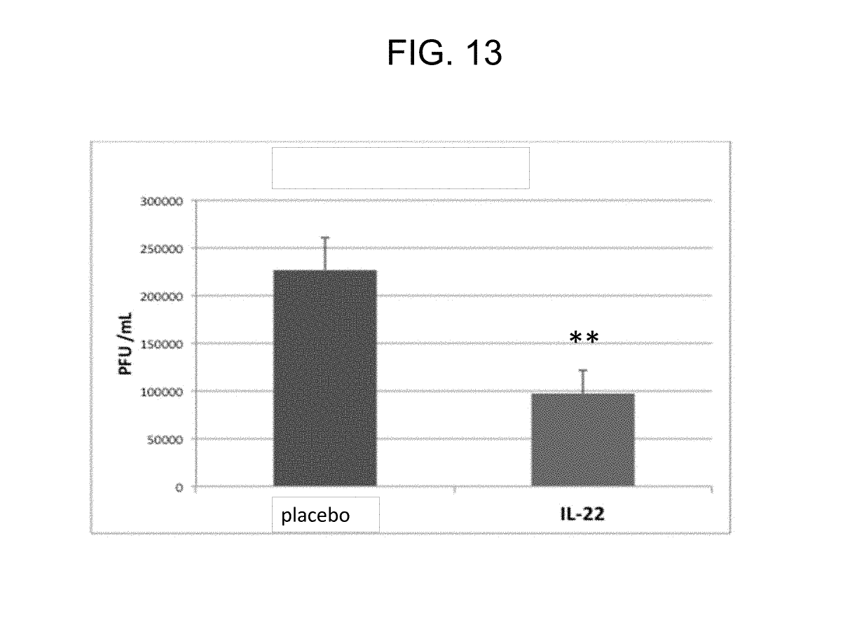

The invention also relates to the prevention of Influenza infection using IL-22. Recombinant IL-22 (2 μg) was administrated intra-nasally to mice (BALBcJRj female/6w-old) at 48 hr (D-2) before the virus inoculation followed by a second administration of 100 ng of IL-22 the same day (D0) of the intranasal viral inoculation (H1N1 WSN 10,000 PFU/mouse). Viral titration was done MDCK cells at 7 days virus post-infection from ground extracts of the left lung of euthanized animals (left lung ground in 600 uL of culture medium). The results shown in FIG. 13 demonstrated a difference in favor of an IL-22 anti-Influenza activity (p=0.0125).

We discovered that IL22 directly inhibit viral infection and the viral entry when cell lines and epithelial primary cells (from the lung) are used and exposed to the influenza viruses. It has been suggested that the upregulation of an antigen function, preferably a B lymphocyte antigen function but also viral antigen stimulation of T cells (isolated ex vivo, exposed to antigen presenting cells expressing IL-22 peptides) may be used as means of regulating immune responses and as such be useful in antiviral therapy. Data in the state of art exist that provides proof that IL22 is not effective in stimulating B or T cells as such lack IL-22 receptors. Such effect can only be exerted via direct interaction of IL-22 with the cells of the mucosa or other tissues as only these cells are capable of responding to IL-22. Our data demonstrate that humans who are not capable of expressing high levels of IL-22 gene when viral antigens are present and as a consequence get infected as demonstrated in the examples with EU (HIV exposed and uninfected individuals) and HIV infected patients. We provide a method of preventing or treating viral infections in individuals who do not have inherent ability of responding with increased IL-22 gene expression when exposed to viral infections.

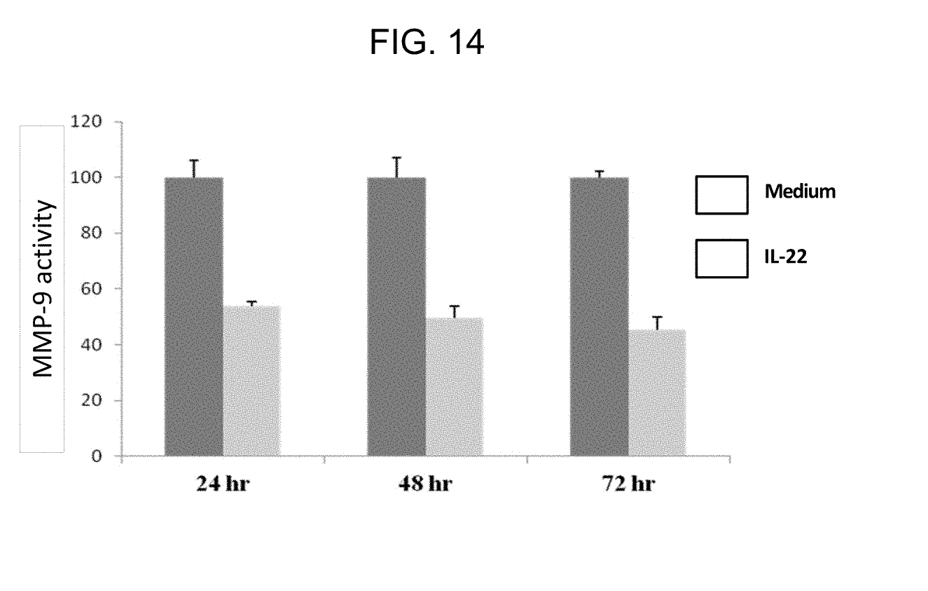

The inventors have also shown that IL-22 decreases MMP-9 activity in activated lung epithelial cells which could be one mechanism of action of IL-22 to prevent/treat Influenza infection. This invention also relates to the use of exogenous recombinant IL-22 for the stimulation of lung epithelium to reduce the pathophysiological consequences of infection by influenza virus including microbial translocation as the permeability of epithelial tissue is decreased, explaining already reported data showing that IL-22 is capable of inhibiting super infections with bacteria in individuals suffering from influenza. One of the consequences of viral infection is acute inflammatory response by the body, whose intensity varies according to parameters such as viral load, intrinsic virulence of infectious virus, and the state of homeostasis within the host. This is particularly true for the highly pathogenic strains derived from avian viruses. This can lead to increases in the epithelial and endothelial permeability of multiple organs. Regarding MMP-9 metalloprotease we have demonstrated its role in these pathophysiological processes, for example in Dengue infection (Luplertiop, Missè et al. 2006). We have demonstrated that IL-22 is able to decrease the activity of MMP-9 in activated lung epithelial cells (FIG. 14).

Other characteristics and advantages of the invention will be given in the following examples and with reference to FIGS. 1 to 14, which represent, respectively:

FIGS. 1A to 1D: HIV infection and HIV genome integration in human genome (IA); HIV envelope (gp120 & gp41) attaching to cell receptors (IB); HIV-cell fusion and capside cell entry (IC); Different steps of the viral envelope attachment to cell receptors (ID);

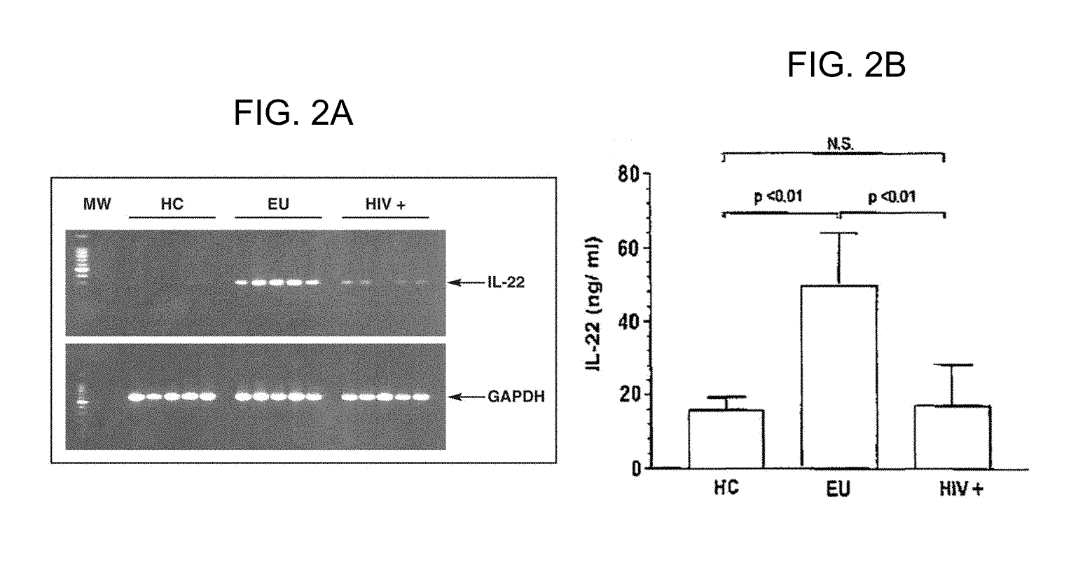

FIGS. 2A to 2B: Comparative IL-22 RT-PCR (A) and IL-22 secreted protein (B) by T-cell from individuals from different cohorts;

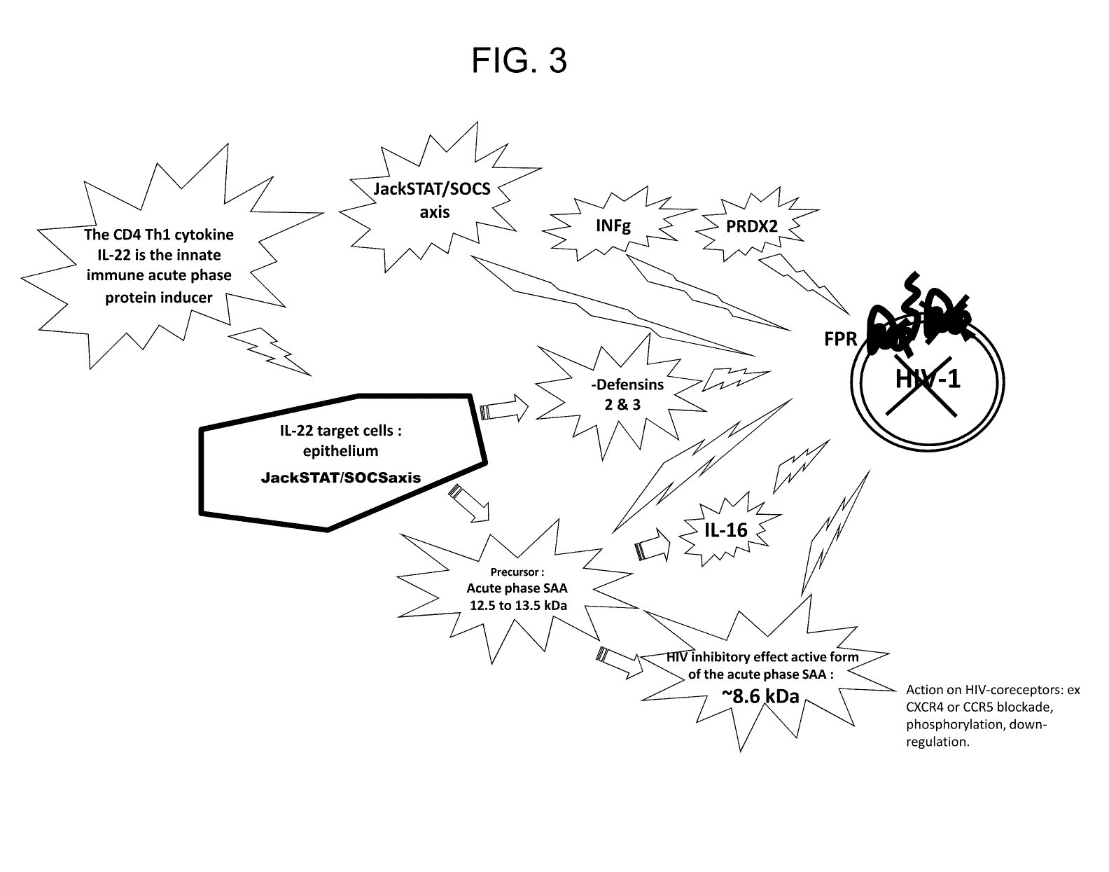

FIG. 3: Inhibitory effects on HIV-I infection of the cascade infection EU;

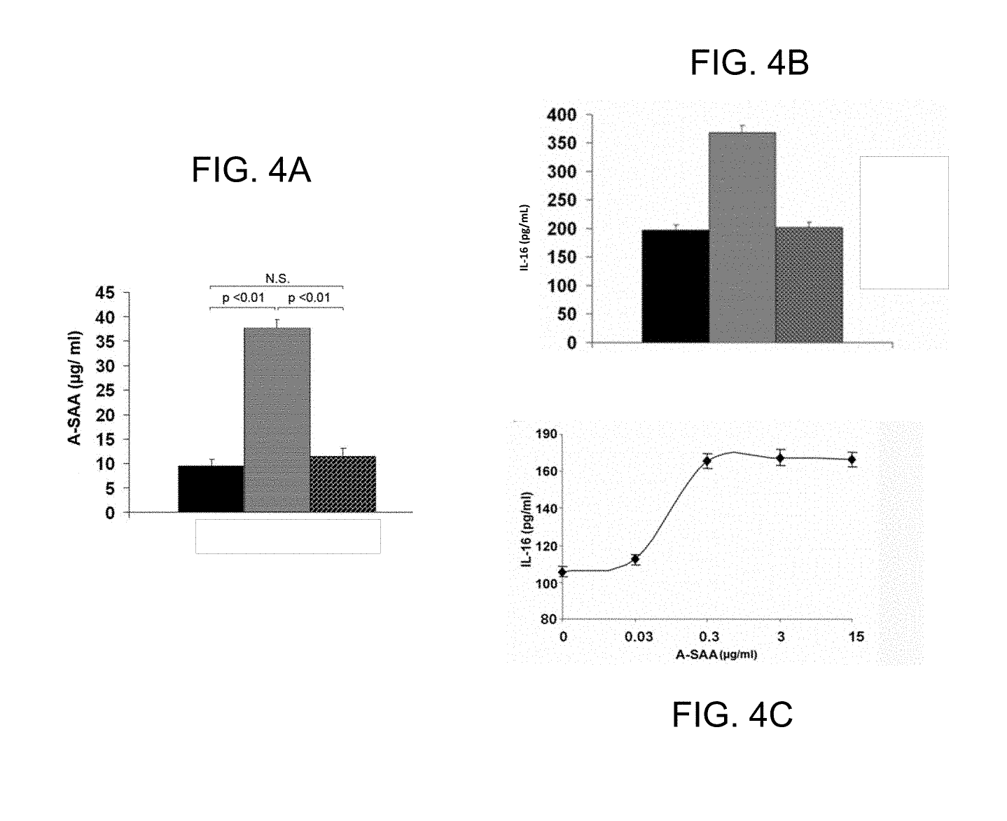

FIGS. 4A to 4C: Serum levels of A-SAA (A) and IL-16 (B) within the three groups and induction of IL-16 by A-SAA (4C);

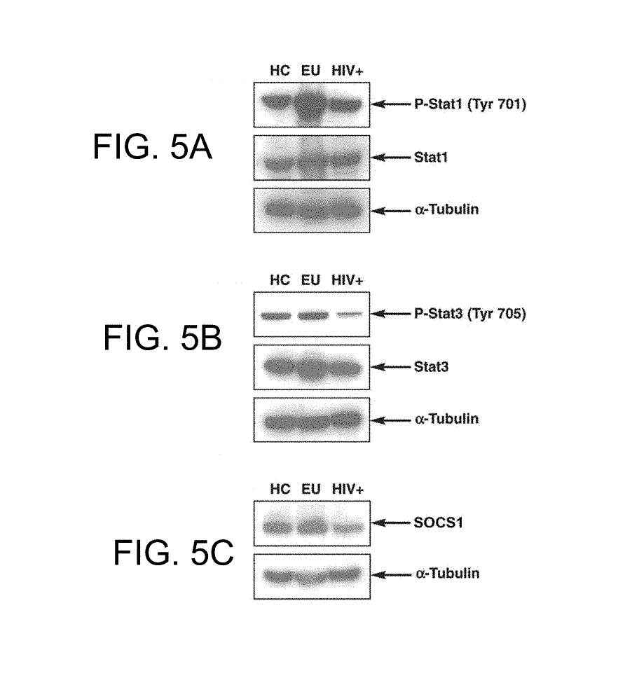

FIGS. 5A to 5C: Stat/SOCS axis expression and activation. Western blot validation of SAGE analysis from individuals from different cohorts (HC: healthy controls; EU:HIV-exposed uninfected and HIV+: HIV-infected);

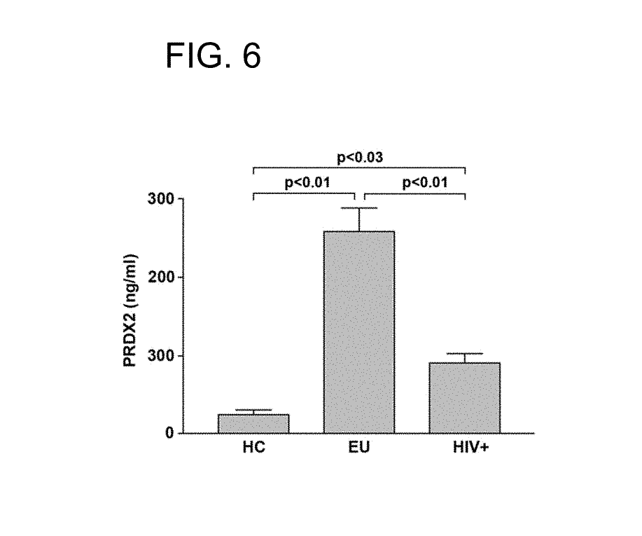

FIG. 6: Serum from HIV-1 exposed uninfected individual contains up-regulated PRDX2 protein, natural killer enhancing factor, compared to sera from HIV+ or HC;

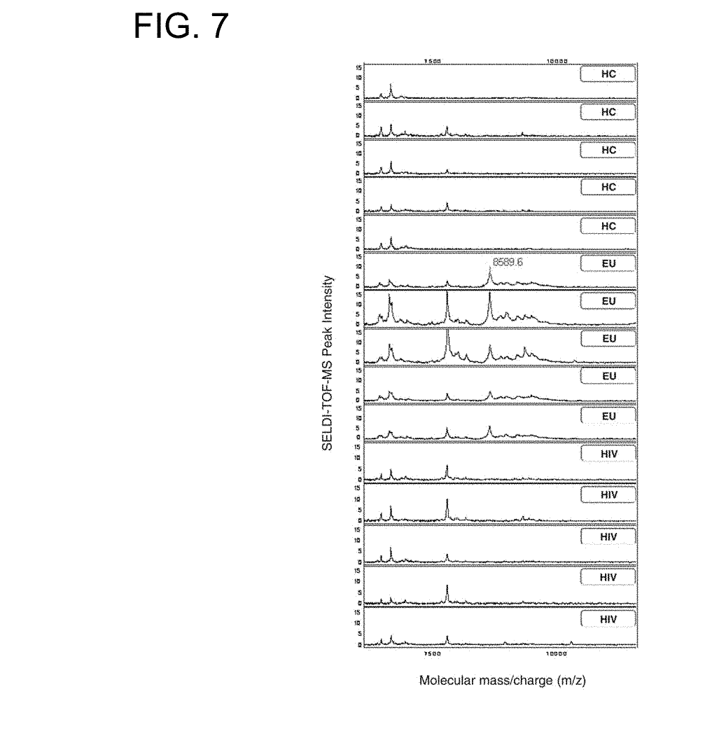

FIG. 7: SELDI-TOF protein profile from individuals from the three groups;

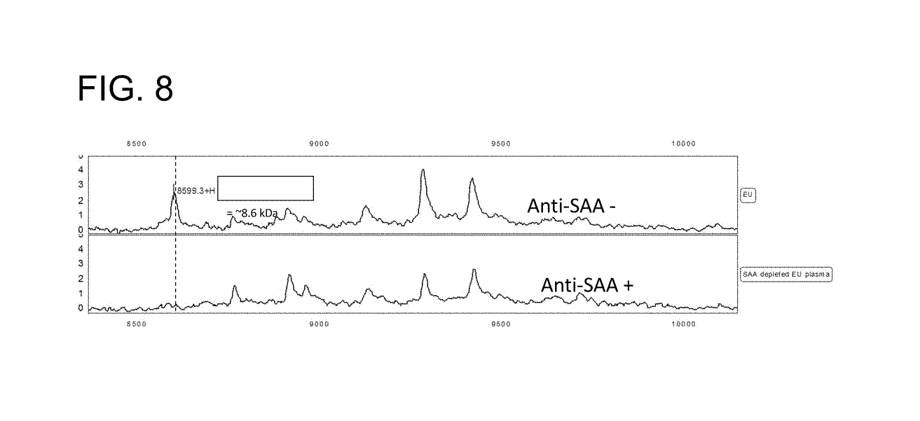

FIG. 8: Identification of 8.6 kDa as a fragment of A-SAA by its depletion using an anti-A-SAA Mab;

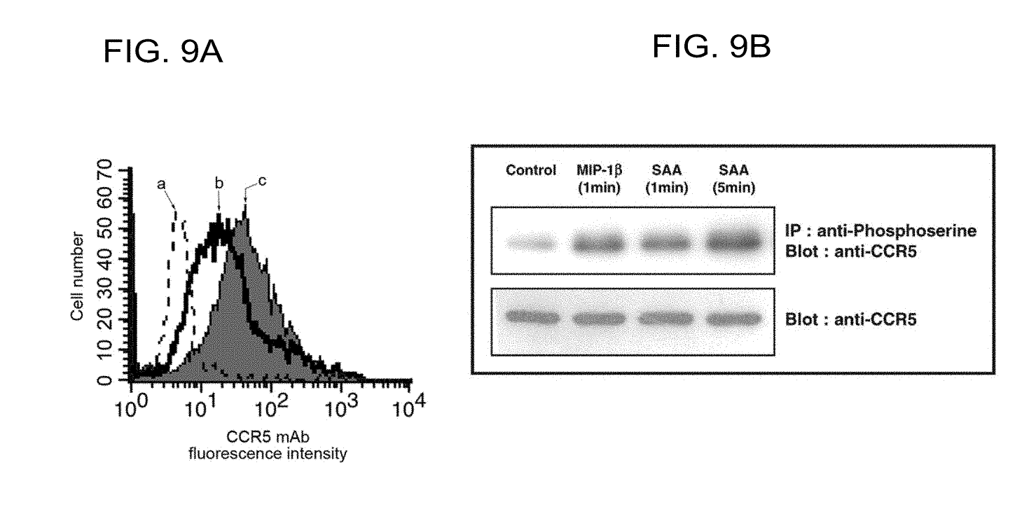

FIGS. 9A to 9B: (9A) CCR5 receptor down modulation induced by the binding of the acute phase A-SAA protein to the FPR receptor cells incubated with an isotype matched control mAb (a), cells pre-incubated with 10 μg/mL of recombinant A-SAA (b) or pre-incubated with culture medium alone (c) were stained with a FITC-conjugated anti-CCR5 mAb; (9B) HIV-I CCR5 coreceptor phosphorylation induced by the binding of the acute phase A-SAA protein to the FPR receptor;

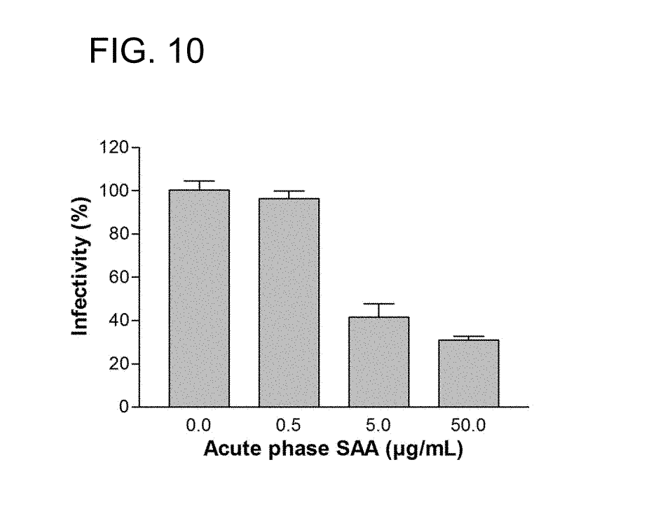

FIG. 10: HIV-I R5 infectivity of immature dendritic cells upon their stimulation with A-SAA acute phase protein;

FIG. 11: Inhibition of Influenza viral entry by IL-22;

FIGS. 12A to 12B: Effect of IL-22 on Influenza viral replication;

FIG. 13: Effect of IL-22 on mice treated with intranasal administration of IL-22 for 48 hrs and infected with H1 N1 virus;

FIG. 14: Effect of IL-22 on MMP-9 activity.

### MATERIALS AND METHODS

### Exposed Uninfected (EU) Individuals Recruitment

HIV exposed but uninfected individuals were enrolled in the study. In each case the ESN was the sexual partner of a HIV infected patient; in each couple a prolonged history of penetrative sexual intercourse without condom (and no other known risk factors) was reported. Inclusion criteria for the EU was a history of multiple unprotected sexual episodes for at least four years with at least four episodes of at-risk intercourse within 4 months prior to the study period. EUs were repeatedly HIV seronegative by culture and RNA virus load methods. HIV-infected individuals and healthy controls were also enrolled in the study. HIV patients and HC were age-and-sex-matched with the EU. All EU, HIV+ and HC individuals had been longitudinally followed for at least 4 years (prior to the study period) by the Department of Infectious Diseases, Santa Maria Annunziata Hospital in Florence. This allowed to exclude from the study ESN and HC in whom sexually transmitted diseases or any other pathology had been reported in that time period. The EU were characterized on the basis of the presence of CCR54.32 alleles; a heterozygous deletion was detected in I individual that was excluded from the study. All EU, HIV patients and low-risk uninfected individuals agreed to donate peripheral blood mononuclear cells.

### EU and HIV-I-Infected Individuals (Table 2)

N couples discordant for HIV-1 serostatus were enrolled. In each case the EU was the sexual partner of an HIV infected patient; in each couple a prolonged history of penetrative sexual intercourse without condom (and no other known risk factors) was reported. The female partner was HIV-1-infected in N couples, whereas the male partner was HIV-1-infected in the remaining N couples. The inclusion criteria for the EU group were a history of multiple unprotected sexual episodes for >5 years with at least 3 episodes of at-risk intercourses within 4 months prior to the study point. Self-administered questionnaires show that the couples reported an average of 34 unprotected sexual episodes/year (range 12 to 50) in the three years previous to the study; vaginal intercourse was the rule and anal sex was not reported by any of the participants in the study. The serostatus of the EU, analyzed by ELISA and Western blot techniques at regular intervals, has always been negative.

In all the infected individuals the diagnosis of HIV-I infection was made during the chronic phase of infection, and thus unprotected sexual intercourses had been initiated long before their diagnosis. Mean CD4 counts of the infected partners at the time of this study was evaluated. All EU, HIV-seropositive, and HC individuals had been longitudinally followed for at least 5 years (prior to the study period) by the Department of Infectious Diseases, Santa Maria Annunziata Hospital in Florence. This allowed us to exclude from the study EU and HC in whom sexually transmitted diseases or any other pathology had been reported in that time period. The EU were characterized on the basis of the presence of CCR5-Δ32 alleles; a heterozygous deletion was detected in I individual. All the enrollees are Caucasians from Toscany region. The ethics committee of the above hospital have approved the research protocols. Written informed consent was obtained from all enrollees, and samples were anonymized and analyzed in a blinded fashion.

### Cells

Proteomic and Transcriptomic comparative studies were carried out on T cells from EU and HIV+ forming discordant couples having frequent unprotected sexual intercourse or invasive drug injection by syringe exchanges. T cells from HC were the controls of these analyses. Peripheral blood mononuclear cells (PBMC), obtained from the 3 cohorts: HC, EU and HIV+ were collected and separated over Ficoll-Hypaque, were cultivated (Yssel, H. and Spits, H, in Current Protocols in Immunology, Chapter 7.19) then T lymphocytes (CD4+ and CD8+) were CD3/CD28 activated and cultivated in RPMI supplemented of 10% of FCS. Briefly, to activate the CD3-TCR complex, 10 μg/mL of anti-CD3, SPV-T3b monoclonal antibody (MAb) was used to coat 24-well plates for 4 hr at 37° C. Subsequently, 106 cells were then deposited in these coated wells in the presence of culture medium (Yssel's medium, Irvine scientific, Santa Ana, Calif.) containing 1% of AB+ human serum and 1 μg/mL of anti-CD28 L293 MAb. Three T cells activation times were respectively done 2, 6 and 18 hr. Activated cells pooled from 5 individuals per cohort (having each an equivalent number of cells and total RNA, Table 2) for T cell gene expression studies that were carried out using the Serial Analysis Gene Expression (SAGE, Velculescu 1995, [24]). Subsequently, a set of total ARN of each individual of the pool was freeze for further use to validate individually the SAGE results. A set of these cells was also used to perform Power and Western blotting analyses (see below). Soluble proteins presents in the plasma of individuals (n=21, Table 2) from 3 cohorts were analysed by SELDITOF Ciphergen™ approach.

Dendritic cell were derived of monocytes from healthy donors. Briefly, a buffy coat was processed to obtain highly purified monocytes that were cultivated in DMEM medium supplemented of 10% of FCS in the presence of 10 ng/mL of IL-4 and 150 ng of GM-CSF (Becton and Dickinson) for 7 days up to obtain well characterized using appropriated MAbs (anti-DC sign, Anti-CDIa, anti-CD83 and anti-CD86 MAbs) also exhibiting the presence of the Formyl peptide receptor-like 1 (FPRLI) (a receptor belonging to the Formyl Peptide receptor (FPR) family) immature Dendritic Cells (iDC). Cells were maintained at 37° C. in a 5% CO2 humid atmosphere.

### Antibodies and Reagents

Recombinant human IL-22, anti-CCR5 polyclonal Ab, anti-human IL-22 polyclonal were purchased from R & D Systems (Oxon, UK), Serum amyloid A (A-SAA) and IL-8 proteins were purchased from Peprotech (Rocky Hill, N.J.), MIP-1β was obtained from (Franqoise Baleux (Pasteur Institute, Paris, France), Anti-IL-8 MAb was purchased from Bender, Anti-CXCR4, Anti-SAA1 and 2 MAb (Biosource), Anti-SAA MAb (Calbiochem), Anti-active Stat-1 polyclonal Ab, anti-Stat1 MAb, Anti-active Stat-3 polyclonal Ab, Anti-Stat-3 pAb, anti-active Stat-5 polyclonal Ab, Anti-Stat-5 MAb was purchased from Becton and Dickinson (Palo Alto, Calif.). The anti-SOCS 3 polyclonal Ab (Santa Cruz laboratories, Santa Cruz, Calif.)

### Plasma Analysis by Protein-Chip SELDI-TOF Approach

Before analysis, plasma samples were centrifuged at 13 000 rpm during 15 min, the pellet was discarded and supernatant was diluted (1:10) in optimized binding buffer (BB: NaCl 0.250 M Hepes 50 mM, pH 7.5). Diluted plasma samples were applied during 1 hr onto previous saturated strong anion exchanger (SAX2) Protein-chips™ by two BB baths of 5 min. Unbound proteins were washed out using successively 3 washes of 5 min with the washing buffer (WB: NaCl IM, Hepes 50 mM, pH 7.5) and a final wash using 5 μL of pure bi-distilled water. The Chip-captured proteins were subsequently air-dried at room temperature (RT) before their covering with a matrix (3,5-dimethoxy-4-hydroxycinnapynic acide (SPA) in 99.9% acetonitril and 0.1% trifluoroacetic acid) to absorb the laser energy. The matrix-prepared samples were dried at RT.). The ionized and desorbed proteins were detected and their molecular masses pointed on the proteogram pies were determined using TOF analysis with the Protein-Chip Biology System II software (PBS II; Ciphergen) and the Ciphergen Peaks software. The mass to charge ratio (m/z) of each captured protein by the chip-surface was determined according to externally calibrated standards: human Angiotensin 1 (1.2965 kilodaltons, kDa), human ACTH (2.9335 kDa), human a-endorphin (3.4650 kDa), bovine insulin (5.7336 kDa), and bovine ubiquitin (8.5648 kDa). Depletion of the Protein of −8.6 kDa from EU Plasma.

Twenty five microliters of magnetic beads (Dynal) washed 3 times with 1 mL of PBS were added of 25 μg of anti-A-SAA (SAA-1 & SAA-2) MAb from Clinisciences concentrated at 100 μg/mL and incubated for 18 hr at 4° C. in orbital shaking. Anti-A-SAA MAb coated beads were subsequently washed 3 times with 1 mL of PBS. Five hundred microliters of EU plasma was then added and incubated at 37° C. during 3 hr under shaking. This plasma supernatant was then reanalysed using the appropriated Ciphergen Chip. Five microliters of EU preincubated with anti-A-SAA 1 & 2 MAb or not were applied and analysed as previously indicated by SELDI-Tof (Ciphergen™). Inhibition of HIV-1 Infection by Recombinant A-SAA Protein

Before HIV-1 infection iDC cells were incubated for 1 hr at the designated concentrations with the acute phase human apolipoprotein serum amyloid A (SSA from Peprotec™) which is an agonist of FPRLI. Subsequently, the cells were infected with HIV-1 ADA or HXB2 at an MOI of 0.1 for 2 hours. The cells were extensively washed and incubated in complete medium. HIV-1 p24 levels were determined by enzyme-linked immunosorbent assays (BeckmanCoulter, France) 4 days after infection.

### Myeloid Immature Dendritic Cells

Human, monocyte-derived, immature dendritic cells (iDC) were generated in vitro as follows: PBMC, obtained from healthy individuals, were isolated by Ficoll-Hypaque density centrifugation and incubated for 30 min at 37° C. in gelatin-coated culture flasks that had been coated with 2% gelatin (for coating, 75 cm2 plastic tissue culture flasks were incubated with 5 mL of 2% gelatin, Sigma-Aldrich, Lyon, France) for 2 hr at 37° C. After removal of gelatin, flasks were incubated upright at 37° C. for 24 hr and washed one with RPMI-1640, supplemented with 2% heat-inactivated fetal calf serum (FCS; Life Technologies, Cergy Pontoise, France), prior to addition of cells), in RPMI-1640/10% FCS. After removal of the non-adherent cells by extensive washing with RPMI/2% FCS, the remaining adherent cells were incubated with 10 mM of EDTA for 5 min at 37° C., collected, washed and cultured in IMDM (Life Technologies), supplemented with 10% FCS, in the presence of 100 ng/mL rGM-CSF and 10 ng/mL rIL-4 (both purchased from BruCells, Brussels, Belgium). After four days of culture, the cells, consisting mainly of iDC, were collected and used in subsequent experiments. Populations of in vitro generated CD1a⁺, CD14⁻, CD83⁻, CD86⁺ and FPRL1⁺ iDC were >97% pure, as determined by immunofluorescence and flow cytometry, using FITC-conjugated mAb purchased from BD/PharMingen, La Jolla, Calif.).

### SAGE Analysis

SAGE was performed as outlined in the detailed protocol by Velculescu et al [Velculescu et al., 1995], obtainable at the URL: WWW.sagenet.org. Only differences in levels of mRNA expression between the three cohorts greater than 10 with a p value <0.01 using the Audie and Claverie algorithm [Audie and Claverie, 1997] were considered.

### Power Blot Analysis

Immunoblot analysis of proteins was carried out as described (www.translab.com/shtml). Briefly, CD3/CD28-stimulated T cells from the 3 cohorts were lysed by the lysis buffer (Tris 10 mM pH 7.4, Na+ orthovanadate 1 mM, SDS 1%−), sonicated and clarified by centrifugation. Proteins were migrated in 5-15% gradient SDS-polyacrylamide gels to detect a wide size range of proteins in one gel. Four hundred micrograms of protein was loaded in long well across the entire width of the gel. This translates into near 15 μg of protein electrophoresed per lane on a standard 25-well gel. Subsequently the gel was transferred to Immobilon-P membrane (Millipore, Bedford, Mass.) overnight. After transfer, membranes were blocked for 1 hr with 5% milk. Subsequently, the membrane was inserted into a Western blotting manifold that isolates 45 channels across the membrane. In each channel, different complex antibody cocktails were added and allowed to hybridize for 1 hr. Following staining, the membranes were washed and hybridised for 30 min with secondary goat anti-mouse horseradish peroxidase (HRP). All antibodies were mouse monoclonal. Membranes were washed and developed with SuperSignal West Pico (Pierce, Santa Clara, Calif.). RT-PCR Analysis

Total RNA, isolated from activated T cells was converted by reverse transcription into cDNA. For each total RNA sample reverse transcription at 42° C. for 50 min, the following reagents were used: 1 μg total RNA and 200 Units Superscript II reverse transcriptase (RT, Gibco-BRL); RT buffer as supplied; 100 mmol/L dithiothreitol (DTT), 40 units of Rnasin (Promega, Madison, Wis., USA); 1.25 mmol/L of each dNTP; and 500 ng of oligo dTs. PCR was performed as follow: 2 μL cDNA; 1.25 mmo/L of each dNTP, 2.5 units Taq polymerase (Promega); 2.5 mmol/L MgCl2, 2.5 μL IO× buffer and 20 pmol of each specific primer pair in a 25 μL total volume. The following specific primers were used: IL-22: SEQ ID No 1 sense 5′-TGACAAGTCCAACTTCCAGCAG-3′, SEQ ID No 2 antisense 5′-TCTGGATATGCAGGTCATCACC-3′; GAPDH: SEQ ID No 3 sense 5′-CCA-CCC-ATG-GCA-AAT-TCC-ATGGCA-3′ and SEQ ID No 4 antisense 5′-TCTAGACGGCAGGTCAGGTCCACC-3′. After preincubation (94° C., 5 min), each PCR sample underwent a 29 cycles amplification regimen of denaturation (94° C., 1 min), primer annealing (56° C., 1 min) and primer extension (72° C., 1 min) with a final extension (72° C., 10 min).

### Western Blotting Analysis

One million of CD3/CD28 activated T cells (as indicated above) from each cohort (HC, EU, and HIV+) were lysed in a 1% NP40 buffer. For each group, equal amounts of protein were electrophoresed under reducing conditions and transferred electrophoretically to nitrocellulose membranes. Membranes were incubated for 30 min in TBS (50 mmol/L NaCl, 20 mmol/L Tris HCl, pH 7.5) containing 5% BSA and 0.1° A) Tween 20 and then incubated overnight at 4° C. with a primary antibody. Proteins were visualized using the ECL system (Amersham Pharmacia Biotech, Piscataway, N.J.). Blots were washed in TBS containing 0.1% Tween 20 and incubated with HRPconjugated goat anti-rabbit or anti-mouse secondary antibody (Amersham Pharmacia Biotech, Piscataway, N.J.). For reblotting with another antibody, filters were stripped as previously described [10].

HIV-1 Coreceptor Phosphorylation Assessing [0061] Immature Dendritic cells were stimulated with MIP-1β or with the acute phase A-SAA (Peprotec™) at the indicated (in FIG. 7) concentrations for the indicated periods of time at 37° C. Then the cells were lysed after 20 min on ice with periodic mixing in lysis buffer (1% Triton X-100, 20 mM Tris HCl pH 8.0, 137 mM NaCl, 15% glycerol, 5 mM EDTA) containing phosphatase inhibitors (1 mM phenylsulfonyl fluoride, 5 μg/mL aprotinin, 5 μg/mL leupeptin, 1 mM sodium orthovanadate, 1 mM EGTA). Cell lysates were precleaned with 30 μL of washed protein A Sepharose beads (15 μL packed beads) at 4° C. for 1 hr and 1 μg of polyclonal anti-phosphoserine antibody (BD) was added to 200 μg cell lysates. The reaction mixture was incubated at 4° C. overnight. The immune complex was captured by adding 50 μL of washed protein A sepharose beads (25 μL packed beads). The reaction mixture was incubated at 4° C. for an additional 2 hours. The beads were spun down (10 sec at 14000 rpm), drained off the supernatant, washed 3 times with ice cold I× IP buffer, then were resuspended in 30 μL 2× Laemli sample buffer and boiled for 5 min to eluate the immune complex. After electrophoresis on 10% SDS-PAGE precast gel (Invitrogen), the proteins were transferred to nitrocellulose membranes. CCR5 was visualized using a polyclonal anti-CCR5 (R & D Systems) and ECL system (Amersham Pharmacia Biotech, Piscataway, N.J.).

### Human Chemokine, Searchlight™ Arrays

Four different plasma from each studied cohort were analysed following the instructions the manufacturer of chemokine Searchlight arrays (Pierce Endogen, Perbio, Boston) for the plasma content in 8 chemokines.

Depletion of the 8.6 kDa MW Protein from EU Plasma

Twenty five microliters of magnetic beads (Dynal, Compiegne, France) were washed 3 times with PBS and added to 25 μg of anti-A-SAA mAb (Biosource, Nivelles, Belgium) and incubated for 1 hr at 4° C. during orbital shaking. Anti-A-SAAmAb-coated beads were subsequently washed 3 times with PBS. EU plasma was then added and incubated at 37° C. during 3 hr under shaking. Five microliters of EU plasma, preincubated in the presence of absence of a neutralizing anti-A-SAA mAb, were analyzed by SELDI-TOF under the conditions described above.

### Measurement of CCR5 Phosphorylation

Myeloid iDC were stimulated with MIP-1β (a kind gift of Franqoise Baleux. Pasteur Institute, Paris, France) or with A-SAA (Peprotech, London, UK), at the indicated concentrations for the indicated periods of time at 37° C. Then the cells were lysed after 20 min on ice with periodic mixing in lysis buffer (1% Triton X-100, 20 mM Tris HCl pH 8.0, 137 mM NaCl, 15% glycerol, 5 mM EDTA) containing phosphatase inhibitors (I mM phenylsulfonyl fluoride, 5 μg/mL aprotinin, 5 μg/mL leupeptin, 1 mM sodium orthovanadate, I mM EGTA). Cell lysates were precleaned with 30 μL of washed protein A Sepharose beads (15 μL packed beads) at 4° C. for I hr and I μg of polyclonal anti-phosphoserine antibody (BD) was added to 200 μg cell lysates. The reaction mixture was incubated at 4° C. overnight. The immune complex was captured by adding 50 μL of washed protein A sepharose beads (25 μL packed beads). The reaction mixture was incubated at 4° C. for an additional 2 hours. The beads were spun down (10 sec at 14000 rpm), drained off the supernatant, washed 3 times with ice cold I× IP buffer, then were resuspended in 30 μL 2× Laemli sample buffer and boiled for 5 min to elute the immune complex. After electrophoresis on I 0% SDS-PAGE precast gel (Invitrogen, Cergy Pontoise, France), the proteins were transferred to nitrocellulose membranes. CCR5 was visualized using a polyclonal anti-CCR5 and ECL system (Amersham Pharmacia Biotech, Piscataway, N.J.).

### HIV-I Infection of iDC

Myeloid iDC cells with incubated various concentration of rA-SAA (PeproTech) for I hr at 37° C. Cells were then incubated with the R5/X4, dual tropic primary isolate HIV-I 4757, at a multiplicity of infection (MOI) of 0.1. After 2 hr of incubation, the cells were washed three times with RPMI-1604/10% FCS and cultured in the same medium. After four days of cultures, the cells were collected and HIV-I p24 levels were determined by commercial ELISA (Beckman-Coulter, Marseilles, France).

### Sera Protein Content

Protein levels in serum were analyzed by highly sensitive Protein Array analysis (Searchlight®: Pierce Endogen, Perbio, Boston), based on detection by chemilummescence.

Influenza experiments: in vitro experiments have been performed on lung cell lines (A549, H1975) and primary cells NHBE (Lonza) then infected using viral strains H1 N1 (WSN) and H3N2 (A/Pan/2007/99). qRT-PCR has been used to quantify viral entry 5 hours post-infection. Effect of IL-22 has been analyzed 48 hours post infection using PFU quantification in cell supernatants. For in vivo experiments, recombinant IL-22 (2 ug) was administrated intra-nasally to mice (BALBcJRj female/6 w-old) at 48 hr (Day-2) before the virus inoculation followed by a second administration of 100 ng of IL-22 the same day (Day0) of the intranasal viral inoculation (H1 N1 WSN 10,000 PFU/mouse). Viral titration was done on MDCK cells at 7 days virus post-infection from the extracts of the left lung of euthanized animals (left lung ground in 600 uL of culture medium).

### Results

The cascade of events initiated by IL-22 that favour the innate host resistance to HIV infection characterizing EU was identified.

Despite being repeatedly exposed to Human Immunodeficiency Type I virus (HIV-I) via sexual or systemic routes certain individuals remain uninfected. To investigate the molecular mechanisms underlying resistance to HIV-1 infection, the inventors have performed a comparative study on CD3/CD28-activated peripheral blood T cells (to enhance cell signaling and gene expression) and plasma (to study their soluble proteins) from cohorts of HIV-1 exposed uninfected individuals (EU), their HIV-1-infected sexual partners and healthy controls (Table 2).

TABLE 2
Immunovirologic and epidemiological characteristics of EU and HIV⁺

| EXPOSED UNINFECTED | | | | | | HIV-INFECTED | | | |
|---|---|---|---|---|---|---|---|---|---|
| INDIVIDUALS | | | | | | PARTNERS | | | |
| | | | | Last at-risk | | | | Viral | |
| | Duration of | | | episode | | | CD4/ | load | |
| | Unprotected | | | (prior to | | CD4/ | CD8 | (copies/ | |
| ID | sex | | sex | enrolment) | | μL | ratio | mL) | therapy |
| 1 | 15 | years | M | <1 | month | 348 | 0.4 | 460 | yes |
| 2 | 8 | years | F | 2 | days | 244 | 0.5 | 24,000 | yes |
| 3 | 6 | years | F | 1 | month | 327 | 0.4 | 400 | yes |
| 4 | 7 | years | M | 1 | day | 328 | 0.3 | 9,420 | yes |
| 5 | 10 | years | M | 1 | month | 916 | 0.4 | 9,440 | no |
| 6 | 12 | years | M | 14 | days | 205 | 0.3 | <50 | yes |
| 7 | 7 | years | F | 2 | months | 16 | 0.2 | 750,000 | yes |
| 8 | 6 | years | M | 3 | days | 632 | 0.5 | 2,070 | yes |
| 9 | 5 | years | F | 2 | months | 101 | 0.3 | 399 | no |
| 10 | 5 | years | F | 3 | months | 424 | 0.3 | 750,000 | no |
| 11 | 5 | years | F | 3 | days | 673 | 0.4 | <50 | yes |
| 12 | 7 | years | M | 2 | days | 452 | 0.4 | 45,800 | no |
| 13 | 10 | years | F | 5 | days | 472 | 0.2 | <50 | yes |
| 14 | 10 | years | M | 10 | days | 1220 | 0.6 | 55,000 | yes |
| 15 | 11 | years | M | 2 | months | 321 | 0.3 | <400 | yes |
| 16 | 5 | years | M | 2 | months | 385 | 0.3 | >750,000 | no |
| 17 | 5 | years | F | 3 | months | 49 | 0.2 | 400 | yes |
| 18 | 7 | years | M | 1 | month | 339 | 0.2 | <400 | yes |
| 19 | 6 | years | F | <1 | month | 458 | 0.3 | 400 | no |
| 20 | 7 | years | F | 1 | month | 327 | 0.3 | 350,000 | no |
| 21 | 8 | years | F | <1 | month | 166 | 0.2 | <50 | yes |

Complementary genomic, proteomic and cell signaling analyses were carried out using Serial Analysis Gene Expression (SAGE), Surface-Enhanced Laser Desorption Tonisation and Time Of Fly Mass Spectrophotometry (SELDI-TOF, Ciphergen™) and

Power blotting™, respectively (see Material and Method Section). Understanding of the genetic and physiology of the Long term non progressors (LTNP) and EU individuals with respect to natural anti-viral mechanisms could provide the basis of the treatment against HIV infection. The inventors have then studied physiopathological mechanisms on the basis of the absence of infection in individuals subject to frequent exposures to HIV in EU individuals.

First results obtained from the high number of gene tags (HC: 21193 tags, EU: 22697 tags, and HIV+: 17 285 tags) of transcriptome analyses by the SAGE method exhibited that in EU were found to overexpress the Th1 IL-22 and SOCS I and that Granzyme B was to underexpress in HIV+ compared to EU and HC cohorts that exhibited similar levels (Table 3A and 3B) these results of course were obtained without having any “a priori” idea.

Sage Data are given in Table 3A which gives the results concerning the differentially expressed genes in activated T cells HIV-I-exposed uninfected individuals, their HIV-I-infected sexual partners and healthy controls. Transcription profiles of T cells, activated via CD3 and CD28 mAb and pooled from the three cohorts of EU, HIV+ and HC (n=21) were analyzed by SAGE. Differences in relative levels of gene expression are indicated as follows: grey indicates up-regulation and light grey indicates downregulation.

Table 3B gives the results concerning power blot analysis

TABLE 3B
Protein level expression

| PROTEIN | HC | ESN | HIV + |
|---|---|---|---|
| STAT3 | 0 | 5 | 0 |

In parallel using Power Blot analysis of proteins from pooled T cells from the 3 cohorts the acute-phase response factor STAT3 was detected. The plasma analyses (using SELDI-TOF (Ciphergen®) approach) from 25 individuals per cohort have shown an expression increase of a soluble protein of a MW of 8.6 kDa,. The results are illustrated by FIGS. 2A and 2B (HC: healthy controls; EU: HIV-exposed uninfected; and HIV+:HIV-infected.

Sage results (Table 3A) revealed that IL-22 is upregulated in EU compared to HIV+ HC groups (FIGS. 2A and 2B).

Taking into account that IL-22 initiates a cascade (FIG. 3) of innate immune response [25] that includes the Jack/STAT/SOCS 1 pathway, SOCS 1 SOCS 3, betadefensins, and the acute phase apolipoprotein serum amyloid A (A-SAA): SAA-1 and SAA2, these data were further developed using different methods to confirm and extend their signification (see Material and Method Section). FIGS. 4A to 4C give the results obtained concerning serum levels of A-SAA (FIG. 4A) and IL-16 (FIG. 4B) within the 3 groups and the induction of IL-16 upon PBMC stimulation with recombinant A-SAA (FIG. 4C). It has been then shown that PBMC stimulated with A-SAA produce IL-16 participates to the reduction to HIV infection.

Sage results also revealed upregulation of STAT1 and SOCS 1 (verified by Western blotting in FIGS. 5A to 5C) and also that PRDX2 is upregulated in EU compared to HIV+ and HC groups (FIG. 6).

Synthesised in the liver and in other tissues as epitheliums of blood vessels, the ASAA is found associated to HDL [26] and HDL-free in the plasma. The A-SAA promoter is highly responsive to inflammatory cytokines such as ILiα, TNFα, IFNα and IL6 that can be induced by LPS. Moreover recently, it has been shown that the Th1 IL-22 cytokine is able to participate to A-SAA expression. These observations have suggested that A-SAA could play a role as an immune innate defense molecule at local sites [27]. Post transductional cleavage of A-SAA produces C-term fragments of approximately 8.5 kDa MW [28, 29].

To identify the −8.6 kDa protein obtained from the SELDI-TOF analysis, a specific anti-A-SAA MAb before the plasma SELDI-TOF profiling was used. The results are given in FIG. 7. As shown, SELDI-TOF (Ciphergen™) protein profiles from individuals from different cohorts (HC:healthy controls; EU HIV-exposed uninfected and HIV+:HIV-infected) exhibit a protein at 8.5896 kDa (−8.6 kDa) that is overexpressed in EU than in other groups.

The upper protein profile (proteogramm) given on FIG. 8 shows the protein of interest at 8599.3 kDa (−8.6 kDA) over expressed among the EU compared to HIV+ discordant couples and HC. The rat anti-huSAA Mab (Clinisciences AHA1011) react with the human isoforms SAA 1 and SAA 2 of the acute phase protein, the apolipoprotein serum amyloid A (SAA). This mAb was able to completely deplete the (−8.6 kDA) protein after immunoprecipiptation from EU plasma indicating as expected that said protein corresponds to the active fragment of an acute phase of SAA.

To correlate the presence of the 8.6 kDa cleavage product of A-SAA in EU plasma with enhanced production their A-SAA levels, serum samples of the individuals from all three groups were analyzed for the presence of A-SAA. Basal serum levels of A-SAA in EU were strongly enhanced, compared to those of HC and HIV+, and thus mirrored the enhanced production of IL-22 by activated T cells of these individuals (FIGS. 4A TO 4C). Taking into account that A-SAA target cells harbouring FPR receptors we therefore analyzed on in vitro generated myeloid iDC express FRP at their surface and that are susceptible to infection with HIV-1 via CCR5. Recombinant A-SAA induced CCR5 phosphorylation in myeloid iDC cells, whereas it also significantly down modulated CCR5 expression on the latter cells (FIGS. 9A and 9B). Importantly, culture of myeloid iDC with the HIV-1, in the presence of increasing concentrations of recombinant A-SAA, resulted in a decreased infection rate in a dose-dependent manner, compared to that of cells infected in the absence of this protein (FIG. 10).

These results allowed to identify IL-22 as a cascade of events triggering factor that favour the innate host resistance to HIV infection characterizing EU. Since IL-22 is able via JAK/STAT to induce the Beta defensins, A-SAA and that A-SAA induces IL-16 secretion, these results clearly depicted this cascade. Moreover, IL-16 [31] and a-Defensin [39] have been shown to decrease HIV-1 infection. Altogether our results show that IL-22 is a starter cytokine helping the innate immune response that provides resistant mechanisms to HIV infection (FIGS. 2A and 2B).

Another exploratory approach was to check some chemokines in plasma from 4 individuals per cohort (EU, HC, HIV+) by using the Serchlight (Perbio™) human chemokine array (Table 4).

GRO-α, MIP-3β, SDFI-β and the gamma chemokine lymphotactin were found to be highly overexpressed in some EU and sometimes in HC, compared to HIV+. The role of these chemokines in HIV infection is not clearly elucidated but Lymphotactin show an anti-HIV activity [1]. However, it is possible to consider the existence of a specific polymorphism of these chemokines that could have an anti-viral effect (individually or combined) of some EU taking into account that in our EU studied cohort we have found an IL-S polymorphism specific of EU.

Additionally the SAGE analysis interestingly shows that Granzyme B was down regulated in HIV+ but maintained in EU and HC individuals, This confirm the observation of the loss of granzyme made in HIV-HAART treated individuals [2, 3]. The inventors have also observed in SAGE analysis that a higher production of IFN-gamma in EU than in HC and HIV+. These cytokine is typically antiviral which has been found in some studies on EU made by others.

Taking into account that the cascade of events was found to induce several and major elements of the innate immunity, the scope of the invention also extends to other viruses and retroviruses than HIV.

It will also be considered that the EU exhibited higher amounts of phosphorylated STAT and that importantly this element is essential to the activity of the “cell anti-viral Factors” (CAF) secreted by CD8 T cells. It has also been shown that HIV+ appears to oose the Granzymes B in comparison with EU and HC. Granzymes B is produced by CD8 T cells and NK to kill infected cells. The STAT-1 dependent production of CAF Granzymes B plays major role in the anti-HIV activity in persons that resist to AIDS development despite their HIV infection. It has also been observed in SAGE analysis that a higher production of IFN-gamma in EU than in HC and HIV+. These cytokine is typically antiviral. INFg induces a better expression of IL-22 receptor in IL-22 target cells.

These cascades elements should be involved not only as element of the resistance to the viral infection but also as element of the resistance to the induced disease.

Results from a comparative analysis of gene transcriptional levels by SAGE of three pooled libraries, prepared from anti-CD3 and anti-CD2S monoclonal antibody (mAb)-activated T cells from all 21 individuals of each cohort showed that expression of transcripts for interleukin-22 (IL-22) was strongly enhanced in EU (differential ratio of 13:1:1 in EU, as compared with their HIV-infected partners and healthy individuals). This observation was validated by RT-PCR analysis, using mRNA isolated from T cells from 5 randomly selected individuals of each cohort that confirmed a strongly enhanced expression of IL-22 mRNA only in activated T cells from EU. The results are given in FIGS. 2A and 2B which concerns activated T cells from EU that overexpress transcripts for IL-22, as compared to those from healthy or HAART-treated, HIV-infected individuals. (FIG. 2A) RT-PCR analysis of total RNA isolated from anti-CD3 and anti-CD2S mAb-activated T cells, obtained from five individuals, who had been randomly chosen from three cohorts consisting of healthy controls (HC), EU and their HAART-treated, HIV-1 infected sexual partners (HIV+), using primers specific for IL-22 and GAPDH as a positive control. These data individually validate results obtained with SAGE in which 20000 tags were analyzed (FIG. 2B). Detection of IL-22 in T cell secreted protein individuals from each cohort, by eELISA. The resulting data were unexpected as inventors were not able to select for the analysis of any particular gene, the samples were subjected to transcriptome analysis capable of identification of amplified genes. IL-22 was the clearly one gene that was identified as dramatically amplified in individuals resistant to being infected by HIV despite repeated risk exposure over long periods of time (unprotected sex).

IL-22, a cytokine, produced by activated CD4 T cells, preferentially of the T helper type 1 phenotype, as well as NK cells, upregulates the production of acute phase proteins, such as acute-phase Serum Amyloid A (A-SAA), a-1 Antichymotrypsin and Haptoglubulin in liver cells and induces the expression of transcripts for β-defensin 2 and 3 in keratinocytes, indicating a role for this cytokine in innate immunity that contribute to host defence against bacterial, fungal and viral infection, including HIV-1.

In order to determine whether the enhanced IL-22 production by EU T cells was reflected in changes in the levels of plasma proteins associated with the biological function of IL-22, a differential protein profile analysis was carried out.

The results are given on FIGS. 4A to 4C which gives serum levels of IL-16 and A-SAA in individuals from each cohort. Serum samples were taken from 15 randomly chosen individuals from each cohort and protein levels of (FIG. 4A) A-SAA and (FIG. 4B) IL-16 were determined by Searchlight® protein array analysis. Results are expressed as mean and SD (n=15 per cohort) and statistical significance was determined by Mann-Whitney non parametric test. (FIG. 4C) Supernatant IL-16 read-out after incubation of primary PBMC for 3 hr at 37° C. in 5% CO2 humid atmosphere with increasing doses of rA-SAA.

As shown in FIGS. 12A and 12B, basal levels of A-SAA in EU individuals was about 4 fold higher than those detected in either healthy controls or HIV-infected individuals, suggesting that high basal level of A-SAA could be a consequence of increased IL-22 expression in EU.

Among the various cytokines and soluble factors tested, serum levels of IL-16 were found to be significantly elevated (>two fold), in EU individuals (FIG. 4B) as compared to either healthy controls or HIV-infected individuals. IL-16, a ligand for CD4, is known to prevent viral entry of both T tropic and M tropic isolates of HIV or SIV secondarily to its capacity to modulate CCR5-mediated signaling by a mechanism of heterologous receptor desensitization. Moreover, IL-16 inhibits viral replication by repressing HIV-1 promoter activity and could therefore be involved in the host defence to HIV infection and replication. The observation that EU sera contain enhanced levels of IL-16 prompted us to examine whether the production of this cytokine could be linked with the functional activity of IL-22 and in particular with the capacity of the latter to induce the production of acute phase proteins. Indeed, stimulation of PBMC with rA-SAA resulted in a significant dose-dependent increase of soluble IL-16 protein production in the cell supernatant with a plateau near to 170 μg/mL (FIG. 4C).

In order to analyse comparatively the soluble protein profiling, plasma samples from all subjects were processed by SELDI-TOF mass spectrometry. Results are given on FIG. 7 which concerns a 8.6 kDa A-SAA-cleaved fragment which is a specific clinical biomarker for EU. (A) Protein profiles of plasma samples from healthy controls (HC), EU and their HAART-treated, HIV-I-infected sexual partners (HIV were determined by SELDI-TOF-MS, using SAX2 Protein Chips. A protein peak with a MW of 8.6 kD was specifically detected in all 25 EU plasma samples tested (indicated by arrow in 5 samples shown), but were absent in the other 50 samples from the other cohorts. (B) A pool of plasma samples from 5 EU was incubated with either a mAb, specific for the 13.5 kDa MW unprocessed precursor of A-SAA, or with an isotype-matched control mAb, coupled to magnetic beads and after removal of the beads re-analyzed by SELDI-TOF-MS. The results show (a) the presence of the −8.6 kDa in the absence of anti-A-SAA treatment and (b) a complete depletion of this peak, the MW of which corresponds to the size of the AA protein, the major metabolic cleavage product of A-SAA; showed the presence of a protein with a molecular weight of 8.6 kDa in plasma from EU, that was absent in all plasma samples from the other cohorts (FIG. 7). A search in Swiss-Prat data bank matched, among the 8 identified proteins, to A-SAA (UniProtKB/Swiss-Prot entry P02735) and its cleavage product of 76 amino terminal residues, corresponding to amyloid protein-A (AA1 or AA2 and their isoforms). To verify the identity of this protein fragment, experiments were carried out to determine whether it was recognized by a monoclonal antibody (mAb) specific for the 13.5 kDa MW unprocessed precursor of A-SAA. Incubation of EU plasma samples with this mAb, bound to magnetic beads, followed by depletion of the mAb-protein complex, resulted in a complete removal of the −8.6 kDa peak from the mass spectrometry protein profile (FIG. 8), demonstrating that it correspond a cleaved product of A-SAA. These results indicate that a −8.6 kDa cleavage product of A-SAA, is specifically present in EU individuals, and it might serve as a specific biomarker.

A-SAA is one of many agonists of a group of formyl peptide receptors (FPR) that belong to the seven membrane domain Gai-protein-coupled receptor family. Activation of FPR modulates the expression and function of Gai-protein-coupled receptors, such as the HIV-1 co-receptors CXCR4 and CCR5, by heterologous receptor desensitization (see for review [Le et al., 2001]). In particular, stimulation of monocytes with A-SAA induces serine phosphorylation of CCR5 which is accompanied by its downregulation from the cell surface and a decreased signaling capacity in response to its natural ligands MI P-1β and RANTES. The results are given on FIG. 10 which shows that A-SAA decreases HIV-1 infection in vitro. (A) Immunoprecipitation and Western blotting analysis show that rA-SAA induces serine phosphorylation of CCR5 on in vitro monocyte-derived immature dendritic cells. MIP-1β has been used as a positive control. Down regulation of CCR5 induced by a 30 min treatment of the cells by 10 μg/mL of rA-SAA. Preincubation of the cells with the indicated concentrations of rA-SAA results in a decrease of infection with the R5/X4, dual tropic, primary isolate HIV-1 4757, as determined by measuring HIV-1 p24 levels using enzyme-linked immunosorbent assays. Results represent mean and SD of 3 independent experiments; Indeed, it was observed that A-SAA was able, not only, to induce CCR5 phosphorylation in dendritic cells derived in vitro from primary monocytes (FIG. 14A), but also CCR5 down modulation. Furthermore, culture of these dendritic cells with the HIV-1 X4/R5 dual tropic 4757 primary isolate, in the presence of rA-SAA resulted in a decreased infection rate as compared that of cells infected in the absence of this protein.

In conclusion, said data identify a biochemical cascade of events that is triggered by IL-22 and appears to have a pivotal role in the innate resistance to HIV-1 infection. Results shown herewith indicate that IL-22 not only induces acute phase proteins such as A-SAA and ˜−defensins, but also initiates the A-SAA-mediated production of IL-16, resulting in phosphorylation and down-regulation of CCR5 and, finally, in a decreased in vitro susceptibility of target cells to infection with primary isolates of HIV. These mechanisms are likely to be important in the development of novel therapeutic and vaccinal approaches for HIV infection.

The results are illustrated by FIGS. 5A to 5C which gives Western blot validation results of SAGE analysis from individuals from different cohorts (HC: healthy controls; EU:HIV-exposed uninfected and HIV+: HIV-infected) showing that the STATs and SOCS are activated in T cells from EU.

Moreover, considering that one of the target cell types of IL-22 are epithelial cells, it appears that IL-22 could induce in endocervix epithelial cells some of the proteins of the innate immune response already published [Wolk et al., 2004]. Endocervix HeLa cell line were incubated with IL-22 that resulted in a dose dependent increase of A-SAA and β-Defensin-2 expression and very interestingly, it was shown for the first time that, IL-16 expression was also increased upon epithelial cells stimulation by IL-22. Thus, the inventors have shown for the first time that IL-16 is induced by cervix epithelial HeLa cell stimulated by IL-22 and that IL-16 production was enhanced by 9 fold upon stimulation of PBMC cells by rA-SAA.

We have evaluated the role of IL-22 in murine models of H1 N1 viral infection. In Vitro data obtained on lung cells lines (A549, H1975), and primary cell cultures NHBE (Lonza) have shown that IL-22 reduces viral infection (H1 N1 (WSN) and H3N2 (A/Pan/2007/99). To study the effect of IL-22 on viral entry, quantifications of viral RNA have been done 5 h after infection of NHBE primary cell cultures and H1975 cell line with H1 N1 (WSN) (FIG.11). Quantification of viral RNA in primary cells NHBE and in H1975 cell line (pre-incubation 48 h with IL-22) 5 hours after infection with H1 N1 (WSN) show an inhibition of Influenza RNA by IL-22.

Effect of IL-22 has been evaluated 48 hr after infection by quantification of PFU in corresponding supernatants (FIGS. 12A and 12B). Quantification of PFU in supernatant of cells A549 pre-incubated for 48 hrs with IL-22 and infected with H1N1 virus (WSN) and incubated for another 48 hrs. IL-22 inhibits significantly influenza viral replication (FIGS. 2A and 2B).

Recombinant IL-22 (2 ug) was administrated intra-nasally to mice (BALBcJRj female/6w-old) at 48 hr (D-2) before the virus inoculation followed by a second administration of 100 ng of IL-22 the same day (D0) of the intranasal viral inoculation (H1N1 WSN 10,000 PFU/mouse). Viral titration was done MDCK cells at 7 days' virus post-infection from ground extracts of the left lung of euthanized animals (left lung ground in 600 uL of culture medium). The results shown in FIG._3 exhibited a difference in favor of an IL-22 anti-Influenza activity (p=0.0125).

Quantification of MMP-9 activity in supernatant of cells stimulated with TNF-α with or without IL-22 show that IL-22 inhibits MMP-9 activity (FIG. 14) which further supports the mechanism of action of IL-22 to alleviate influenza infection. We favor the hypothesis that the protective role of 1L-22 in bacterial and viral infection is rather due to its positive effect on epithelium, injury due to the pathogen rather than to an antibacterial effector mechanism per se. Thus, by limiting the alteration of the epithelial barrier instigated by the pathogen, 1L-22 might decrease the level of pathogen invasion into the tissues.

### REFERENCES

1. Ranki, A., Mattinen, S., Yarchoan, R., Broder, S., Ghrayeb, J., Landevirta, J., Krohn, K. (19S9) T-cell response towards HIV in infected individuals with and without zidovudine therapy, and in HIV-exposed sexual partners. Aids 3, 63-9.

2. Clerici, M., Berzofsky, J. A., Shearer, G. M., Tacket, C. 0. (1991) Exposure to human immunodeficiency virus (HIV) type I indicated by HIV-specific T helper cell responses before detection of infection by polymerase chain reaction and serum antibodies [ corrected]. J Infect Dis 164, 17S-S2.

3. Clerici, M., Giorgi, J. V., Chou, C. C., Gudeman, V. K., Zack, J. A., Gupta, P., Ho, H. N., Nishanian, P. G., Berzofsky, J. A., Shearer, G. M. (1992) Cell-mediated immune response to human immunodeficiency virus (HIV) type 1 in seronegative homosexual men with recent sexual exposure to HIV-1. J. Infect Dis 165, 1012-9.

4. Clerici, M., Levin, J. M., Kessler, H. A., Harris, A., Berzofsky, J. A., Landay, A. L., Shearer, G. M. (1994) HIV-specific T-helper activity in seronegative health care workers exposed to contaminated blood. Jama 271, 42-6.

5. Fowke, K. R., Nagelkerke, N. J., Kimani, J., Simonsen, J. N., Anzala, A. 0., Bwayo, J. J., MacDonald, K. S., Ngugi, E. N., Plummer, F. A. (1996) Resistance to HIV-1 infection among persistently seronegative prostitutes in Nairobi, Kenya. Lancet 34S, 1347-51.

6. Rowland-Jones, S., Sutton, J., Ariyoshi, K., Dong, T., Gotch, F., McAdam, S., Whitby, D., Sabally, S., Gallimore, A., Corrah, T. (1995) HIV-specific cytotoxic T cells in HIV-exposed but uninfected Gambian women. Nat Med I, 59-64.

7. Kaul, R., Rowland-Jones, S. L., Kimani, J., Dong, T., Yang, H. B., Kiama, P., Rostron, T., Njagi, E., Bwayo, J. J., MacDonald, K. S., McMichael, A. J., Plummer, F. A. (2001) Late seroconversion in HIV-resistant Nairobi prostitutes despite pre-existing HIV specific CD8+ responses. J. Clin Invest 107, 341-9.

S. Clerici, M., Clark, E. A., Polacino, P., Axberg, I., Kuller, L., Casey, N. I., Morton, W. R., Shearer, G. M., Benveniste, R. E. (1994) T-cell proliferation to subinfectious SIV correlates with lack of infection after challenge of macaques. Aids S, 1391-5.

9. Samson, M., Libert, F., Doranz, B. J., Rucker, J., Liesnard, C., Farber, C. M., Saragosti, S., Lapoumeroulie, C., Cognaux, J., Forceille, C., Muyldermans, G., Verhofstede, C., Burtonboy, G., Georges, M., Imai, T., Rana, S., Yi, Y., Smyth, R. J., Coltman, R. G., Dams, R. W., Vassart, G., Parmentier, M. (1996) Resistance to HIV-1 infection in caucasian individuals bearing mutant alleles of the CCR-5 chemokine receptor gene. Nature 3S2, 722-5.

10. Kostrikis, L. G., Huang, Y., Moore, J. P., Wolinsky, S. M., Zhang, L., Guo, Y., Deutsch, L., Phair, J., Neumann, A. U., Ho, D. D. (199S) A chemokine receptor CCR2 allele delays HIV-1 disease progression and is associated with a CCR5 promoter mutation. Nat Med 4, 350-3.

11. Paxton, H., Pins, M., Denton, G., McGonigle, A. D., Meisner, P. S., Phair, J. P. (1995) Comparison of CD4 cell count by a simple enzyme-linked immunosorbent assay using the TRAx CD4 test kit and by flow cytometry and hematology. Clin Diagn Lab Immunol 2, 104-14.

12. Quillent, C., Oberlin, E., Braun, J., Rousset, D., Gonzalez-Canali, G., Metais, P., Montagnier, L., Virelizier, J. L., Arenzana-Seisdedos, F., Beretta, A. (1998) HIV-I-resistance phenotype conferred by combination of two separate inherited mutations of CCR5 gene. Lancet 351, 14-S. 12

13. Hsueh, F. W., Walker, C. M., Blackbourn, D. J., Levy, J. A. (1994) Suppression of HIV replication by CD8+ cell clones derived from HIV-infected and uninfected individuals. Cell Immunol 159, 271-9.

14. Mackewicz, C. E., Blackboum, D. J., Levy, J. A. (1995) CD8+ T cells suppress human immunodeficiency virus replication by inhibiting viral transcription. Proc Natl Acad Sci USA 92, 230S-12.

15. Trabattoni, D., Caputo, S. L., Maffeis, G., Vichi, F., Biasin, M., Pierotti, P., Fasano, F., Saresella, M., Franchini, M., Ferrante, P., Mazzotta, F., Clerici, M. (2004) Human alpha Defensin in HIV-Exposed But Uninfected Individuals. J Acquir Immune De.fie Syndr 35, 455-463.

16. Stranford, S. A., Skumick, J., Louria, D., Osmond, D., Chang, S. Y., Sninsky, J., Ferrari, G., Weinhold, K., Lindquist, C., Levy, J. A. (1999) Lack of infection in HIV exposed individuals is associated with a strong CD8(+) cell noncytotoxic anti-HIV response. Proc Natl Acad Sci USA 96, 1030-5.

17. Levy, J. A., Mackewicz, C. E., Barker, E. (1996) Controlling HIV pathogenesis: the role of the noncytotoxic anti-HIV response of CD8+ T cells. Immunol Today 17, 217-24.

1 S. Furci, L., Scarlatti, G., Burastero, S., Tambussi, G., Colognesi, C., Quillent, C., Longhi, R., Loverro, P., Borgonovo, B., Gaffi, D., Carrow, E., Malnati, M., Lusso, P., Siccardi, A. G., Lazzarin, A., Beretta, A. (1997) Antigen-driven C-C chemokinemediated HIV-1 suppression by CD4(+) T cells from exposed uninfected individuals expressing the wild-type CCR-5 allele. J Exp Med 1S6, 455-60.

19. Lizeng, Q., Nilsson, C., Sourial, S., Andersson, S., Larsen, 0., Aaby, P., Ehnlund, M., Bjorling, E. (2004) Potent neutralizing serum immunoglobulin A (IgA) in human immunodeficiency virus type 2-exposed IgG-seronegative individuals. J Viral 7S, 7016-22.

20. Mazzoli, S., Trabattoni, D., Lo Caputo, S., Piconi, S., Ble, C., Meacci, F., Ruzzante, S., Salvi, A., Semplici, F., Longhi, R., Fusi, M. L., Tofani, N., Biasin, M., Villa, M. L., Mazzotta, F., Clerici, M. (1997) HIV-specific mucosa! and cellular immunity in HIV seronegative partners of HIV-seropositive individuals. Nat Med 3, 1250-7.

21. Beyrer, C., Artenstein, A. W., Rugpao, S., Stephens, H., Vancott, T. C., Robb, M. L., Rinkaew, M., Birx, D. L., Khamboonruang, C., Zimmerman, P. A., Nelson, K. E., Natpratan, C. (1999) Epidemiologic and biologic characterization of a cohort of human immunodeficiency virus type 1 highly exposed, persistently seronegative female sex workers in northern Thailand. Chiang Mai HEPS Working Group. J Infect Dis 179, 59-67.

22. Belec, L., Ghys, P. D., Hocini, H., Nkengasong, J. N., Tranchot-Diallo, J., Diallo, M. 0., Ettiegne-Traore, V., Maurice, C., Becquart, P., Matta, M., Si-Mohamed, A., Chomont, N., Coulibaly, I. M., Wiktor, S. Z., Kazatchkine, M. D. (2001) Cervicovaginal secretory antibodies to human immunodeficiency virus type 1 (HIV-1) that block viral transcytosis through tight epithelial barriers in highly exposed HIV-1-seronegative African women. J Infect Dis 1S4, 1412-22.

23. Scott-Algara, D., Truong, L. X., Versmisse, P., David, A., Luong, T. T., Nguyen, N. V., Theodorou, I., Barre-Sinoussi, F., Pancino, G. (2003) Cutting edge: increased NK cell activity in HIV-I-exposed but uninfected Vietnamese intravascular drug users. J Immunol 171, 5663-7.

24. Velculescu, V. E., Zhang, L., Vogelstein, B., Kinzler, K. W. (1995) Serial analysis of gene expression. Science 270, 4S4-7.

25. Wolk, K., Kunz, S., Witte, E., Friedrich, M., Asadullah, K., Sabat, R. (2004) IL-22 increases the innate immunity of tissues. Immunity 21, 241-54. 13

26. Jensen, L. E., Whitehead, A. S. (199S) Regulation of serum amyloid A protein expression during the acute-phase response. Biochem J 334 (Pt 3), 4S9-503.

27. Uhlar, C. M., Whitehead, A. S. (1999) Serum amyloid A, the major vertebrate acutephase reactant. Eur J Biochem 265, 501-23.

2S. Skinner, M. (1992) Protein AA/SAA. J Intern Med 232, 513-4.

29. Ham, D., Skoryna, S. C. (2004) Generation of amyloid A protein by the cell lines from amyloid-susceptible and -resistant mice. Scand J Immunol 59, 117-22.

30. He, R., Sang, H., Ye, R. D. (2003) Serum amyloid A induces IL-8 secretion through a G proteincoupled receptor, FPRL1/LXA4R. Blood 101, 1572-SI.

31. Richardson, R. M., Tokunaga, K., Marjoram, R., Sata, T., Snyderman, R. (2003) Interleukin-S-mediated heterologous receptor internalization provides resistance to HIV-1 infectivity. Role of signal strength and receptor desensitization. J Biol Chem 27S, 15S67-73.

32. Fu, H., Bylund, J., Karlsson, A., Pellme, S., Dahlgren, C. (2004) The mechanism for activation of the neutrophil NADPH-oxidase by the peptides formyl-MetLeu-Phe and Trp-Lys-Tyr-Met-Val-Met differs from that for interleukin-8. Immunology 112, 201-10.

33. Chang, T. L., Mosoian, A., Pine, R., Klotman, M. E., Moore, J. P. (2002) A soluble factor(s) secreted from CD8(+) T lymphocytes inhibits human immunodeficiency virus type 1 replication through STATI activation. J Viral 76, 569-SI.

34. Le, Y., Li, B., Gong, W., Shen, W., Hu, J., Dunlop, N. M., Oppenheim, J. J., Wang, J. M. (2000) Novel pathophysiological role of classical chemotactic peptide receptors and their communications with chemokine receptors. Immunol Rev 177, 1S5-94.

35. Le, Y., Shen, W., Li, B., Gong, W., Dunlop, N. M., Wang, J. M. (1999) A new insight into the role of “old” chemotactic peptide receptors FPR and FPRL1: down-regulation of chemokine receptors CCR5 and CXCR4. Forum (Genova) 9, 299-314.

36. Le, Y., Wetzel, M. A., Shen, W., Gong, W., Rogers, T. J., Henderson, E. E., Wang, J. M. (2001) Desensitization of chemokine receptor CCR5 in dendritic cells at the early stage of differentiation by activation of formyl peptide receptors. Clin Immunol 99, 365-72.

37. Shen, W., Li, B., Wetzel, M. A., Rogers, T. J., Henderson, E. E., Su, S. B., Gong, W., Le, Y., Sargeant, R., Dimitrov, D. S., Oppenheim, J. J., Wang, J. M. (2000) Downregulation of the chemokine receptor CCR5 by activation of chemotactic formyl peptide receptor in human monocytes. Blood 96, 2SS7-94.

3S. Li, B. Q., Wetzel, M. A., Mikovits, J. A., Henderson, E. E., Rogers, T. J., Gong, W., Le, Y., Ruscetti, F. W., Wang, J. M. (2001) The synthetic peptide WKYMVm attenuates the function of the chemokine receptors CCR5 and CXCR4 through activation offormyl peptide receptor-like 1. Blood 97, 2941-7.

39. Quinones-Maten, M. E., Lederman, M. M., Feng, Z., Chakraborty, B., Weber, J., Rangel, H. R., Marotta, M. L., Mirza, M., Jiang, B., Kiser, P., Medvik, K., Sieg, S. F., Weinberg, A. (2003) Human epithelial beta-defensins 2 and 3 inhibit HIV-1 replication. Aids 17, F39-4S.

40. Greco, G., Mackewicz, C., Levy, J. A. (1999) Sensitivity of human immunodeficiency virus infection to various alpha, beta and gamma chemokines. J Gen Viral 80 (Pt 9), 2369-73.

41. Trabattoni, D., Piconi, S., Biasin, M., Rizzardini, G., Migliorino, M., Seminari, E., Boasso, A., Piacentini, L., Villa, M. L., Maserati, R., Clerici, M. (2004) Granuledependent mechanisms of lysis are defective in CD8 T cells of HIV-infected, antiretroviral therapytreated individuals. Aids 18, 859-69. 14

42. Trabattoni, D., Fossati, S., Biasin, M., Boasso, A., Rizzardini, G., Maseratti, R., Clerici, M. (2002) Functional analysis of HIV-specific cytotoxic T lymphocytes in antiviral-treated- and -naive patients: a preliminary report. J Biol Regul Homeost Agents 16, 25-9.

43. O'Hara, R., Murphy, E. P., Whitehead, A. S., FitzGerald, 0., Bresnihan, B. (2000) Acute-phase serum amyloid A production by rheumatoid arthritis synovial tissue. Arthritis Res 2, 142-4.

44. Sipe, J. D., Johns, M. A., Ghezzi, P., Knapschaefer, G. (1988) Modulation of serum amyloid A gene expression by cytokines and bacterial cell wall components. Adv Exp Med Biol 243, 193-201.

45. Steel, D. M., Whitehead, A. S. (1994) The major acute phase reactants: C-reactive protein, serum amyloid P component and serum amyloid A protein. Immunol Today 15, 81-8.

46. Kisilevsky, R. (1991) Serum amyloidA (SAA), a protein without a function: some suggestions with reference to cholesterol metabolism. Med Hypotheses 35, 337-41.

47. Bausserman, L. L., Bernier, D. N., McAdam, K. P., Herbert, P. N. (1988) Serum amyloid A and high density lipoproteins during the acute phase response. Eur J Clin Invest 18, 619-26.

48. Malle, E., Steinmetz, A., Raynes, J. G. (1993) Serum amyloid A (SAA): an acute phase protein and apolipoprotein. Atherosclerosis 102, 131-46.

49. Liang, J. S., Sipe, J. D. (1995) Recombinant human serum amyloid A (apoSAAp) binds cholesterol and modulates cholesterol flux. J Lipid Res 36, 37-46.

50. Meek, R. L., Urieli-Shoval, S., Benditt, E. P. (1994) Expression of apolipoprotein serum amyloid A mRNA in human atherosclerotic lesions and cultured vascular cells: implications for serum amyloidA function. Proc Natl AcadSci USA 91, 3186-90.

51. Nakayama, T., Sonoda, S., Urano, T., Yamada, T., Okada, M. (1993) Monitoring both serum amyloid protein A and C-reactive protein as inflammatory markers in infectious diseases. Clin Chem 39, 293-7.

52. Su, S. B., Gong, W., Gao, J. L., Shen, W., Murphy, P. M., Oppenheim, J. J., Wang, J. M. (1999) A seven-transmembrane, G protein-coupled receptor, FPRLI, mediates the chemotactic activity of serum amyloid A for human phagocytic cells. J Exp Med 189, 395-402.

53. Badolato, R., Wang, J. M., Stornello, S. L., Ponzi, A. N., Duse, M., Musso, T. (2000) Serum amyloid A is an activator of PMN antimicrobial functions: induction of degranulation, phagocytosis, and enhancement of anti-Candida activity. J Leukoc Biol 67, 381-6.

54. Le, Y., Gong, W., Tiffany, H. L., Tumanov, A., Nedospasov, S., Shen, W., Dunlop, N. M., Gao, J. L., Murphy, P. M., Oppenheim, J. J., Wang, J. M. (2001) Amyloid (beta)42 activates a G-protein-coupled chemoattractant receptor, FPR-like-1. J Neurosci 21, RC123.

55. de Paulis, A., Florio, G., Prevete, N., Triggiani, M., Fiorentino, I., Genovese, A., Marone, G. (2002) HIV-1 envelope gp41 peptides promote migration of human Fe epsilon RI+ cells and inhibit IL-13 synthesis through interaction with formyl peptide receptors. J Immunol 169, 4559-67.

56. Hartt, J. K., Liang, T., Sahagun-Ruiz, A., Wang, J. M., Gao, J. L., Murphy, P. M. (2000) The HIV-1 cell entry inhibitor T-20 potently chemoattracts neutrophils by specifically activating the N-formylpeptide receptor. Biochem Bio phys Res Commun 272, 699-704.

57. Wild, C. T., Shugars, D. C., Greenwell, T. K., McDanal, C. B., Matthews, T. J. (1994) Peptides corresponding to a predictive alpha-helical domain of human 15 immunodeficiency virus type 1 gp41 are potent inhibitors of virus infection. Proc Natl Acad Sci USA 91, 9770-4.

58. Kilby, J. M., Hopkins, S., Venetta, T. M., DiMassimo, B., Cloud, G. A., Lee, J. Y., Alldredge, L., Hunter, E., Lambert, D., Bolognesi, D., Matthews, T., Johnson, M. R., Nowak, M. A., Shaw, G. M., Saag, M. S. (1998) Potent suppression of HIV-1 replication in humans by T-20, a peptide inhibitor of gp41-mediated virus entry. Nat Med 4, 1302-7.

59. Aramori, I., Ferguson, S. S., Bieniasz, P. D., Zhang, J., Cullen, B., Cullen, M. G. (1997) Molecular mechanism of desensitization of the chemokine receptor CCR-5: receptor signaling and internalization are dissociable from its role as an HIV-1 co-receptor. Embo J 16, 4606-16.

60. Oppermann, M., Mack, M., Proudfoot, A. E., Olbrich, H. (1999) Differential effects of CC chemokines on CC chemokine receptor 5 (CCR5) phosphorylation and identification of phosphorylation sites on the CCR5 carboxyl terminus. J Biol Chem 274, 8875-85.

61. Olbrich, H., Proudfoot, A. E., Oppermann, M. (1999) Chemokine-induced phosphorylation of CC chemokine receptor 5 (CCR5). J Leukoc Biol 65, 281-5.

62. Dumoutier, L., Lejeune, D., Colau, D., Renauld, J. C. (2001) Cloning and characterization of IL-22 binding protein, a natural antagonist of IL-10-related T cell derived inducible factor/IL-22. J Immunol 166, 7090-5.

63. Gurney, A. L. (2004) IL-22, a Thl cytokine that targets the pancreas and select other peripheral tissues. Int Immunopharmacol 4, 669-77.

64. Nagem, R. A., Colau, D., Dumoutier, L., Renauld, J. C., Ogata, C., Polikarpov, I. (2002) Crystal structure of recombinant human interleukin-22. Structure (Camb) 10, 1051-62. [0164] 65. Xie, M. H., Aggarwal, S., Ho, W. H., Foster, J., Zhang, Z., Stinson, J., Wood, W. I., Goddard, A. D., Gurney, A. L. (2000) Interleukin (IL)-22, a novel human cytokine that signals through the interferon receptorrelated proteins CRF2-4 and IL-22R. J Biol Chem 275, 31335-9.

66. Kotenko, S. V., Izotova, L. S., Mirochnitchenko, O. V., Esterova, E., Dickensheets, H., Donnelly, R. P., Pestka, S. (2001) Identification of the functional interleukin-22 (IL-22) receptor complex: the IL-10R2 chain (IL-IORbeta) is a common chain of both the IL-10 and IL-22 (1L-1 0-related T cell-derived inducible factor, IL-TIF) receptor complexes. J Biol Chem 276, 2725-32.

67. Dumoutier, L., Leemans, C., Lejeune, D., Kotenko, S. V., Renauld, J. C. (2001) Cutting edge: STAT activation by IL-1 9, IL-20 and mda-7 through IL-20 receptor complexes of two types. J Immunol 167, 3545-9.

68. Lejeune, D., Dumoutier, L., Constantinescu, S., Kruijer, W., Schuringa, J. J., Renauld, J. C. (2002) Interleukin-22 (IL-22) activates the JAK/STAT, ERK, JNK, and p38 MAP kinase pathways in a rat hepatoma cell line. Pathways that are shared with and distinct from IL-10. J Biol Chem 277, 33676-82.

69. Radaeva, S., Sun, R., Pan, H. N., Hong, F., Gao, B. (2004) Interleukin 22 (IL-22) plays a protective role in T cell-mediated murine hepatitis: IL-22 is a survival factor for hepatocytes via STAT3 activation. Hepatology 39, 1332-42.

1-8. (canceled)

12-13. (canceled)

14. The use of IL-22 to prevent or repair epithelial cell damages caused by pathogens to prevent pathogen adhesion to the mucosa and its subsequent crossing through the epithelium.

15. The use of IL-22 according to claim 14 wherein the pathogens comprise HIV and influenza viruses.

16. The use of IL-22 as a method to protect and repair epithelium barrier.

17. The use of IL-22 as a method to activate epithelial cells to produce proteins to prevent or treat infection.

18. The use of IL-22 according to claim 17, wherein said proteins comprise soluble SAA, bacterial chemotactic peptide fMLF, at least one protein of an innate immune response from the Jack STAT pathway, SOCS3, beta defensines (2 and 3) and acute phase apolipoprotein serum amyloid Ac or A-SAA or a fragment thereof, SOCS 1 and STATS.

19. Pharmaceutical compositions for preserving, protecting and repairing epithelium for prevention and treatment of infections.

20. The pharmaceutical compositions of claim 19, wherein the pharmaceutical compositions prevent and treat HIV-infection and influenza infection.

21. The pharmaceutical compositions of claim 19, for administration by the oral, intramuscular, intravenous or mucosal route.

22. The pharmaceutical compositions of claim 19, wherein for oral administration, the pharmaceutical compositions are presented in the form of tablets, pills, capsules, drops, patch or spray.

23. The pharmaceutical compositions of claim 19, wherein for administration by injection, the pharmaceutical compositions are under the form of solution for injection by the intravenous, subcutaneous, or intramuscular route produced from sterile or sterilisable solution, or suspension or emulsion, and for mucosal applications under the form of gels.

24. Method to decrease MMP9 in infected tissues of individuals to protect and repair epithelium and therefore treat viral infections.

25. The method of claim 24, wherein the individuals are deficient in ability of increasing IL-22 gene expression.


SEQUENCE LISTING (5 sequences)

SEQ ID NO: 1 - DNA, 22 bp, Artificial sequence
  Primer
tgacaagtccaacttccagcag

SEQ ID NO: 2 - DNA, 22 bp, Artificial sequence
  Primer
tctggatatgcaggtcatcacc

SEQ ID NO: 3 - DNA, 24 bp, Artificial sequence
  Primer
ccacccatggcaaattccatggca

SEQ ID NO: 4 - DNA, 24 bp, Artificial sequence
  Primer
tctagacggcaggtcaggtccacc

SEQ ID NO: 5 - protein, 18 aa, Artificial sequence
  Peptide
TRPLYSTYRMETTRPVAL